(19) United States
(12) Patent Application Publication
(10) Pub. No.: US 2010/0184814 A1
(43) Pub. Date: Jul. 22, 2010

(54) Ester Derivatives of Losartan, Methods for Their Preparation, and Uses Thereof

(76) Inventor: Jianmin Wang, Blacksburg, VA (US)

Correspondence Address: NEW RIVER VALLEY INTELLECTUAL PROPERTY LAW, P.O. BOX 10944, BLACKSBURG, VA, 24062, US

(21) Appl. No.: 12/356,612

(22) Filed: Jan. 21, 2009

(51) Int. Cl. A61K 31/4155; C07D 403/08; A61P 9/12

(52) U.S. Cl. 514/381; 548/253

(57) ABSTRACT

The present invention relates to compounds represented by the formula (I) as prodrugs of the formula (II) (Losartan) and their salts, solvates, hydrates, polymorphs, optical isomers, enantiomers, and pharmaceutical compositions comprising these compounds and methods of preparing them and administering them for the treatment of circulatory diseases such as hypertension, congestive heart failure, angina, and the like.

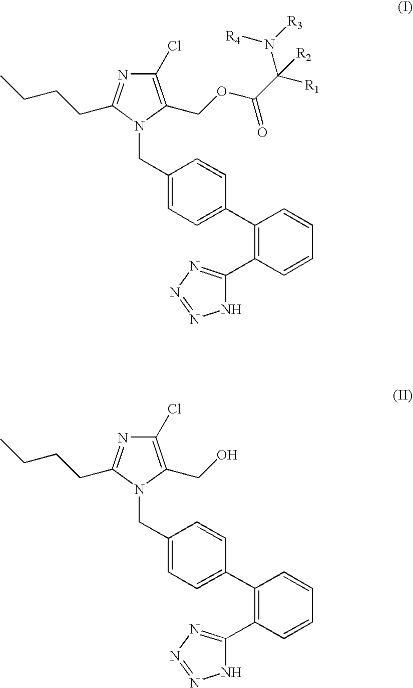

### BACKGROUND OF THE INVENTION

1. Field of the Invention

The present invention relates to a series of compounds and pharmaceutical compositions comprising one or more of these compounds and methods of administering them for the treatment of circulatory diseases such as hypertension, congestive heart failure, angina, and the like.

2. Description of Related Art

Hypertension, or high blood pressure (HBP), is the most important cardiovascular risk factor worldwide, contributing to one half of the coronary heart disease and approximately two thirds of the cerebrovascular disease burdens. In the United States, the estimated prevalence of hypertension (defined as systolic blood pressure [BP]≧140 mm Hg and/or diastolic BP≧90 mm Hg and/or receiving antihypertensive medication) derived from the 1999-2000 Third National Health and Nutrition Examination Survey was 59 million US adults. Due to the epidemic of overweight and obesity occurring in the United States during the past 2 decades, hypertension has been one of the major issues in general health care.

It has been studied and confirmed that by acting on an angiotensin II receptor on the cell membrane, Angiotensin II causes vasoconstriction and elevates blood pressure. Angiotensin II receptor antagonists have been effective therapeutic drugs for treatment of circulatory diseases such as hypertension and the like.

Losartan is an angiotensin II receptor antagonist drug used mainly to treat high blood pressure and it was the first angiotensin II receptor antagonist to be marketed. It is currently marketed by Merck & Co. under the trade name Cozaar. Losartan is a selective, competitive Angiotensin II receptor type 1 (AT₁) receptor antagonist, reducing the end organ responses to angiotensin II. Losartan administration results in a decrease in total peripheral resistance (afterload) and cardiac venous return (preload). All of the physiological effects of angiotensin II, including stimulation of release of aldosterone, are antagonized in the presence of Losartan. Reduction in blood pressure occurs independently of the status of the renin-angiotensin system. As a result of Losartan dosing, plasma renin activity increases due to removal of the angiotensin II feedback.

As with all angiotensin II receptor antagonists, Losartan is indicated for the treatment of hypertension. Losartan may also delay progression of diabetic nephropathy and is also indicated for the reduction of renal disease progression in patients with type 2 diabetes, hypertension and microalbuminuria (>30 mg/24 hours) or proteinuria (>900 mg/24 hours). Although angiotensin II receptor antagonists are not usually considered first-line, because of the proven effectivity and lower costs of thiazide diuretics and beta blockers, Losartan may be used first-line in patients with increased cardiovascular risk. Studies have demonstrated that Losartan was significantly superior to Atenolol in the primary prevention of adverse cardiovascular events (myocardial infarction or stroke), with a significant reduction in cardiovascular morbidity and mortality for a comparable reduction in blood pressure.

Losartan has been found to downregulate the expression of transforming growth factor beta (TGF-β) types I and II receptors in the kidney of diabetic rats, which may partially account for its nephroprotective effects. Effects on TGF-β expression may also account for its potential efficacy in Marfan syndrome and Duchenne muscular dystrophy (DMD)—Losartan has been shown to prevent aortic aneurysm and certain pulmonary complications in a mouse model of the disease.

But Losartan has a half-life of 1-2 hours with bioavailability of 25-35%. Its short half-live and low bioavailability limit its broader use in treatment of hypertension, diabetic nephropathy, renal disease in patients with type 2 diabetes, microalbuminuria, proteinuria and other circulatory diseases. In looking for long lasting and more effective antihypertension agents than Losartan, there is a need to modify Losartan to improve its clinical utility. The present invention provides novel compounds superior as a prodrug of Losartan for treatment of circulatory diseases such as hypertension and the like.

### SUMMARY OF THE INVENTION

One aspect of the present invention is to provide prodrugs of Losartan as novel anti-hypertension agents, or pharmaceutically acceptable salts thereof.

The present invention also provides pharmaceutical compositions comprising a pharmaceutically acceptable carrier and a therapeutically effective amount of at least one of the compounds of the present invention or a pharmaceutically acceptable salt thereof.

The present invention provides a method for treating hypertension diseases and the like comprising administering to a host in need of such treatment a therapeutically effective amount of at least one of the compounds of the present invention or a pharmaceutically acceptable salt thereof.

The present invention provides the method of preparing novel ester derivatives of Losartan for use in therapy.

The present invention provides the use of novel ester derivatives of Losartan for the manufacture of a medicament for the treatment of hypertension diseases and the like.

These and other features, which will become apparent during the following detailed description, have been achieved by the inventors' discovery that one or more compounds of formula (I):

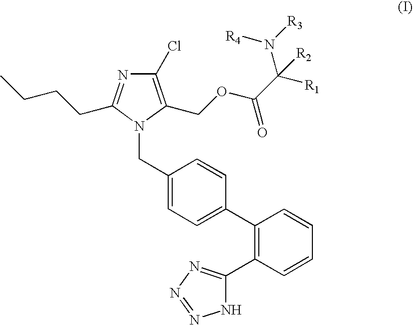

or stereoisomers, optical isomers, enantiomers, solvates, hydrates, polymorphs, or pharmaceutically acceptable salts thereof, wherein R₁, R₂, R₃ and R₄ are defined below, are effective anti-hypertension agents.

### DETAILED DESCRIPTION OF VARIOUS EMBODIMENTS OF THE INVENTION

Thus, in a first embodiment, the present invention provides novel compounds of formula (I):

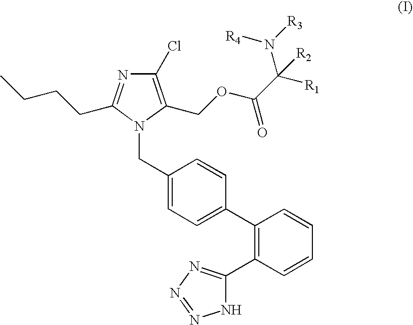

or optical isomers, enantiomers, stereoisomers, solvates, hydrates, polymorphs, or pharmaceutically acceptable salts thereof, wherein R₁, R₂, R₃ and R₄ are each independently, or one or more together form, a group selected from H, C₁₋₈ alkyl, C₁₋₈ alkenyl, C₆₋₁₀ aryl, C₅₋₁₀ heteroaryl, C₃₋₁₀ cycloalkyl, and C₃₋₁₀ heterocycloalkyl.

In another embodiment, the present invention provides compounds of formula (I) wherein, when each of R₁, R₂, R₃ and R₄ independently, or one or more together, comprises an alkyl, alkenyl, aryl, heteroaryl, cycloalkyl, or heterocycloalkyl group, or then the alkyl, alkenyl, aryl, heteroaryl, cycloalkyl, or heterocycloalkyl group is optionally substituted with one or more substituents selected from the group consisting of acyl, alkylamino, amino, aminocarbonyl, acylamino, acyloxy, carboalkoxy, carboxy, carboxyamido, hydroxyl, C₁₋₅ alkyl, C₂₋₄ alkenyl, C₃₋₆ cycloalkyl, C₃₋₆ heterocycloalkyl, C₆ aryl, C₅₋₈ heteroaryl, alkoxy, aryloxy, oxo, and guanidino.

In another embodiment, the present invention provides compounds of formula (1) wherein, R₁, R₂, R₃ and R₄ are each independently, or one or more together comprise, a hydrogen atom or a C₁₋₆ alkyl, and as the C₁₋₆ alkyl, for example, methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituents selected from the group consisting of amino, aminocabonyl, acylamino, acyloxy, carbamoyl, carboalkoxy, carboxy, carboxyamido, hydroxyl, thio, alkylthio, alkoxy, aryloxy, oxo, and guanidino.

In another embodiment, the present invention provides compounds of formula (I) wherein, R₁ and R₂ together form C₃₋₆ cycloalkyl or C₃₋₆ heterocycloalkyl as the C₃₋₆ cycloalkyl or C₃₋₆ heterocycloalkyl, for example, cyclopropyl, cyclobutyl, cyclopentyl, cyclohexyl when R₃ and R₄ are each independently a hydrogen atom or a C₁₋₆ alkyl, and as the C₁₋₆ alkyl, for example, methyl, ethyl, n-propyl, isopropyl, n-butyl, isobutyl, sec-butyl, tert-butyl, n-pentyl, isopentyl, neopentyl, n-hexyl, isohexyl, 1,1 dimethylbutyl, 2,2-dimethylbutyl, 3,3-dimethylbutyl, 2-ethylpropyl which are optionally substituted with one or more substituents selected from the group consisting of amino, aminocabonyl, acylamino, acyloxy, carbamoyl, carboalkoxy, carboxy, carboxyamido, hydroxyl, thio, alkylthio, alkoxy, aryloxy, oxo, and guanidine.

In another embodiment, the present invention provides compounds of formula (1) wherein, R₃ and R₄ together form C₃₋₆ heterocycloalkyl as C₃₋₆ heterocycloalkyl, for example, aziridinyl, azetidinyl, pyrrolidinyl, piperidinyl when R₁ and R₂ are each independently a hydrogen atom or a C₁₋₆ alkyl, and as the C₁₋₆ alkyl, for example, methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituents selected from the group consisting of amino, aminocabonyl, acylamino, acyloxy, carbamoyl, carboalkoxy, carboxy, carboxyamido, hydroxyl, thio, alkylthio, alkoxy, aryloxy, oxo, and guanidino.

In another embodiment, the present invention provides compounds of formula (1) wherein, when R₁, R₃ and R₄ are each a hydrogen atom, R₂ is independently selected from H, CH₃, —CH₂CH₃, —CH(R₅)R₆, —CH₂CH(R₅)R₆, wherein R₅ and R₆ are independently selected from C₁₋₄ alkyl, and as the C₁₋₄ alkyl, for example, methyl, ethyl, n-propyl, isopropyl, n-butyl, isobutyl, sec-butyl, tert-butyl which are optionally substituted with one or more substituents selected from the group consisting of amino, aminocabonyl, acylamino, acyloxy, carbamoyl, carboalkoxy, carboxy, carboxyamido, hydroxyl, thio, alkylthio, alkoxy, aryloxy, oxo, and guanidino.

In another embodiment, the present invention provides compounds of formula (I) wherein, R₁, R₃ and R₄ are each a hydrogen atom, and R₂ is independently selected from H, —CH₃, —CH₂CH₃, —CH(CH₃)CH₃, —CH₂CH(CH₃)CH₃, —CH(CH₃)CH₂CH₃, —CH₂CH₂CONH₂, —CH₂CONH₂, —CH₂CH₂COOH, —CH₂COOH, —SCH₃, —CH₂SH, —CH₂OH, —CH(OH)CH₃, —CH₂CH₂SCH₃, —CH₂C₆H₅, —CH₂C₆H₄(OH), —CH₂CH₂CH₂CH₂NH₂,

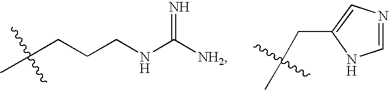

and

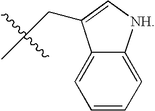

In another embodiment, the present invention provides compounds of formula (I), wherein the compound is selected from the compounds of the Examples.

In another embodiment, the present invention provides pharmaceutical compositions comprising a pharmaceutically acceptable carrier and a therapeutically effective amount of a compound of the present invention or a pharmaceutically acceptable salt thereof.

In another embodiment, the present invention provides a method for treating hypertension comprising administering to a patient in need thereof a therapeutically effective amount of a compound of the present invention, or a pharmaceutically acceptable salt thereof.

The present invention further provides a method for treating disorders selected from hypertension, congestive heart failure, angina, and the like.

In yet another embodiment, the present invention provides novel ester derivatives of Losartan for use in therapy.

In another embodiment, the present invention provides the use of novel alpha-amino ester derivatives of Losartan for the manufacture of a medicament for the treatment of hypertension.

Further still, in another embodiment, the present invention is directed to a process of preparing a compound of formula (I) comprising the steps of reacting a compound of Formula III with a compound of Formula II:

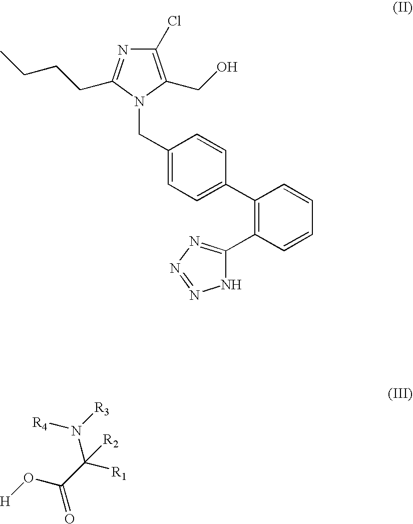

by esterification with or without deprotection methods to obtain the compound of formula (I).

In another embodiment, the present invention is related to a prodrug compound having a particular structure, which is converted to compound (II) (Losartan), for example in the living body, is superior in safety and has extremely superior properties as a pharmaceutical agent, as evidenced by providing longer half-life of Losartan for long lasting hypertensive action, possible stable control of blood pressure for a long time and the like.

In another embodiment, the present invention is related to a compound selected from the group consisting of:
1. (S)-(1-((2′-(2H-tetrazol-5-yl)biphenyl-4-yl)methyl)-2-butyl-4-chloro-1H-imidazol-5-yl)methylpyrrolidine-2-carboxylate;
2. (R)-(1-((2′-(2H-tetrazol-5-yl)biphenyl-4-yl)methyl)-2-butyl-4-chloro-1H-imidazol-5-yl)methylpyrrolidine-2-carboxylate;
3. (S)-(1-((2′-(2H-tetrazol-5-yl)biphenyl-4-yl)methyl)-2-butyl-4-chloro-1H-imidazol-5-yl)methyl piperidine-2-carboxylate;
4. (R)-(1-((2′-(2H-tetrazol-5-yl)biphenyl-4-yl)methyl)-2-butyl-4-chloro-1H-imidazol-5-yl)methyl piperidine-2-carboxylate;
5. (S)-(1-((2′-(2H-tetrazol-5-yl)biphenyl-4-yl)methyl)-2-butyl-4-chloro-1H-imidazol-5-yl)methyl azepane-2-carboxylate;
6. (R)-(1-((2′-(2H-tetrazol-5-yl)biphenyl-4-yl)methyl)-2-butyl-4-chloro-1H-imidazol-5-yl)methyl azepane-2-carboxylate;
7. (1-((2′-(2H-tetrazol-5-yl)biphenyl-4-yl)methyl)-2-butyl-4-chloro-1H-imidazol-5-yl)methyl 1-aminocyclopentanecarboxylate;
8. (1-((2′-(2H-tetrazol-5-yl)biphenyl-4-yl)methyl)-2-butyl-4-chloro-1H-imidazol-5-yl)methyl (1-dimethylamino)cyclopentanecarboxylate;
9. (1-((2′-(2H-tetrazol-5-yl)biphenyl-4-yl)methyl)-2-butyl-4-chloro-1H-imidazol-5-yl)methyl (1-methylamino)cyclopentanecarboxylate;
10. (1-((2′-(2H-tetrazol-5-yl)biphenyl-4-yl)methyl)-2-butyl-4-chloro-1H-imidazol-5-yl)methyl (1-dimethylamino)cyclohexanecarboxylate;
11. (1-((2′-(2H-tetrazol-5-yl)biphenyl-4-yl)methyl)-2-butyl-4-chloro-1H-imidazol-5-yl)methyl (1-methylamino)cyclohexanecarboxylate;
12. 1-((2′-(2H-tetrazol-5-yl)biphenyl-4-yl)methyl)-2-butyl-4-chloro-1H-imidazol-5-yl)methyl 2-methyl-2-(pyrrolidin-1-yl)propanoate;
13. (S)-(1-((2′-(2H-tetrazol-5-yl)biphenyl-4-yl)methyl)-2-butyl-4-chloro-1H-imidazol-5-yl)methyl 2-(pyrrolidin-1-yl)propanoate;
14. (R)-(1-((2′-(2H-tetrazol-5-yl)biphenyl-4-yl)methyl)-2-butyl-4-chloro-1H-imidazol-5-yl)methyl 2-(pyrrolidin-1-yl)propanoate;
15. (1-((2′-(2H-tetrazol-5-yl)biphenyl-4-yl)methyl)-2-butyl-4-chloro-1H-imidazol-5-yl)methyl 2-(dimethylamino)acetate;
16. (1-((2′-(2H-tetrazol-5-yl)biphenyl-4-yl)methyl)-2-butyl-4-chloro-1H-imidazol-5-yl)methyl 2-(methylamino)acetate;
17. (S)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
18. (R)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
19. (S)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
20. (S)-(1-((2′-(2H-tetrazol-5-yl)biphenyl-4-yl)methyl)-2-butyl-4-chloro-1H-imidazol-5-yl)methyl 2-(dimethylamino)-5-guanidinopentanoate;
21. (R)-(1-((2′-(2H-tetrazol-5-yl)biphenyl-4-yl)methyl)-2-butyl-4-chloro-1H-imidazol-5-yl)methyl 2-(dimethylamino)-5-guanidinopentanoate;
22. (S)-((1-((2′-(2H-tetrazol-5-yl)biphenyl-4-yl)methyl)-2-butyl-4-chloro-1H-imidazol-5-yl)methoxy)-3-(methylamino)-4-oxobutanoic acid;
23. (R)-((1-((2′-(2H-tetrazol-5-yl)biphenyl-4-yl)methyl)-2-butyl-4-chloro-1H-imidazol-5-yl)methoxy)-3-(methylamino)-4-oxobutanoic acid;
24. (S)-((1-((2′-(2H-tetrazol-5-yl)biphenyl-4-yl)methyl)-2-butyl-4-chloro-1H-imidazol-5-yl)methoxy)-3-(dimethylamino)-4-oxobutanoic acid;
25. (R)-((1-((2′-(2H-tetrazol-5-yl)biphenyl-4-yl)methyl)-2-butyl-4-chloro-1H-imidazol-5-yl)methoxy)-3-(dimethylamino)-4-oxobutanoic acid;
26. (S)-1-((1-((2′-(2H-tetrazol-5-yl)biphenyl-4-yl)methyl)-2-butyl-4-chloro-1H-imidazol-5-yl)methyl)-4-ethyl 2-(methylamino)succinate;
27. (R)-1-((1-((2′-(2H-tetrazol-5-yl)biphenyl-4-yl)methyl)-2-butyl-4-chloro-1H-imidazol-5-yl)methyl)-4-ethyl 2-(methylamino)succinate;
28. (S)-1-((1-((2′-(2H-tetrazol-5-yl)biphenyl-4-yl)methyl)-2-butyl-4-chloro-1H-imidazol-5-yl)methyl)-4-ethyl 2-(dimethylamino)succinate;
29. (R)-1-((1-((2′-(2H-tetrazol-5-yl)biphenyl-4-yl)methyl)-2-butyl-4-chloro-1H-imidazol-5-yl)methyl)-4-ethyl 2-(dimethylamino)succinate;
30. (S)-(1-((2′-(2H-tetrazol-5-yl)biphenyl-4-yl)methyl)-2-butyl-4-chloro-1H-imidazol-5-yl)methyl 3-mercapto-2-(methylamino)propanoate;
31. (R)-(1-((2′-(2H-tetrazol-5-yl)biphenyl-4-yl)methyl)-2-butyl-4-chloro-1H-imidazol-5-yl)methyl 3-mercapto-2-(methylamino)propanoate;
32. (S)-(1-((2′-(2H-tetrazol-5-yl)biphenyl-4-yl)methyl)-2-butyl-4-chloro-1H-imidazol-5-yl)methyl 3-mercapto-2-(dimethylamino)propanoate;
33. (R)-(1-((2′-(2H-tetrazol-5-yl)biphenyl-4-yl)methyl)-2-butyl-4-chloro-1H-imidazol-5-yl)methyl 3-mercapto-2-(dimethylamino)propanoate;
34. (S)-(1-((2′-(2H-tetrazol-5-yl)biphenyl-4-yl)methyl)-2-butyl-4-chloro-1H-imidazol-5-yl)methyl 4-amino-2-(methylamino)-4-oxobutanoate;
35. (R)-(1-((2′-(2H-tetrazol-5-yl)biphenyl-4-yl)methyl)-2-butyl-4-chloro-1H-imidazol-5-yl)methyl 4-amino-2-(methylamino)-4-oxobutanoate;
36. (S)-(1-((2′-(2H-tetrazol-5-yl)biphenyl-4-yl)methyl)-2-butyl-4-chloro-1H-imidazol-5-yl)methyl 4-amino-2-(dimethylamino)-4-oxobutanoate;
37. (R)-(1-((2′-(2H-tetrazol-5-yl)biphenyl-4-yl)methyl)-2-butyl-4-chloro-1H-imidazol-5-yl)methyl 4-amino-2-(dimethylamino)-4-oxobutanoate;
38. (S)-(1-((2′-(2H-tetrazol-5-yl)biphenyl-4-yl)methyl)-2-butyl-4-chloro-1H-imidazol-5-yl)methyl 5-amino-2-(methylamino)-5-oxopentanoate;
39. (R)-(1-((2′-(2H-tetrazol-5-yl)biphenyl-4-yl)methyl)-2-butyl-4-chloro-1H-imidazol-5-yl)methyl 5-amino-2-(methylamino)-5-oxopentanoate;
40. (S)-(1-((2′-(2H-tetrazol-5-yl)biphenyl-4-yl)methyl)-2-butyl-4-chloro-1H-imidazol-5-yl)methyl 5-amino-2-(dimethylamino)-5-oxopentanoate;
41. (R)-(1-((2′-(2H-tetrazol-5-yl)biphenyl-4-yl)methyl)-2-butyl-4-chloro-1H-imidazol-5-yl)methyl 5-amino-2-(dimethylamino)-5-oxopentanoate;
42. (S)-5-((1-((2′-(2H-tetrazol-5-yl)biphenyl-4-yl)methyl)-2-butyl-4-chloro-1H-imidazol-5-yl)methoxy)-4-(methylamino)-5-oxopentanoic acid;
43. (R)-5-((1-((2′-(2H-tetrazol-5-yl)biphenyl-4-yl)methyl)-2-butyl-4-chloro-1H-imidazol-5-yl)methoxy)-4-(methylamino)-5-oxopentanoic acid;
44. (S)-5-((1-((2′-(2H-tetrazol-5-yl)biphenyl-4-yl)methyl)-2-butyl-4-chloro-1H-imidazol-5-yl)methoxy)-4-(dimethylamino)-5-oxopentanoic acid;
45. (R)-5-((1-((2′-(2H-tetrazol-5-yl)biphenyl-4-yl)methyl)-2-butyl-4-chloro-1H-imidazol-5-yl)methoxy)-4-(dimethylamino)-5-oxopentanoic acid;
46. (S)-1-((1-((2′-(2H-tetrazol-5-yl)biphenyl-4-yl)methyl)-2-butyl-4-chloro-1H-imidazol-5-yl)methyl)-5-methyl 2-(methylamino)pentanedioate;
47. (R)-1-((1-((2′-(2H-tetrazol-5-yl)biphenyl-4-yl)methyl)-2-butyl-4-chloro-1H-imidazol-5-yl)methyl)-5-methyl 2-(methylamino)pentanedioate;
48. (S)-1-((1-((2′-(2H-tetrazol-5-yl)biphenyl-4-yl)methyl)-2-butyl-4-chloro-1H-imidazol-5-yl)methyl)-5-methyl 2-(dimethylamino)pentanedioate;
49. (R)-1-((1-((2′-(2H-tetrazol-5-yl)biphenyl-4-yl)methyl)-2-butyl-4-chloro-1H-imidazol-5-yl)methyl)-5-methyl 2-(dimethylamino)pentanedioate;
50. (S)-(1-((2′-(2H-tetrazol-5-yl)biphenyl-4-yl)methyl)-2-butyl-4-chloro-1H-imidazol-5-yl)methyl 3-(1H-imidazol-2-yl)-2-(methylamino)propanoate;
51. (R)-(1-((2′-(2H-tetrazol-5-yl)biphenyl-4-yl)methyl)-2-butyl-4-chloro-1H-imidazol-5-yl)methyl 3-(1H-imidazol-2-yl)-2-(methylamino)propanoate;
52. (S)-(1-((2′-(2H-tetrazol-5-yl)biphenyl-4-yl)methyl)-2-butyl-4-chloro-1H-imidazol-5-yl)methyl 2-(dimethylamino)-3-(1H-imidazol-2-yl)propanoate;
53. (R)-(1-((2′-(2H-tetrazol-5-yl)biphenyl-4-yl)methyl)-2-butyl-4-chloro-1H-imidazol-5-yl)methyl 2-(dimethylamino)-3-(1H-imidazol-2-yl)propanoate;
54. (2S,3S)-(1-((2′-(2H-tetrazol-5-yl)biphenyl-4-yl)methyl)-2-butyl-4-chloro-1H-imidazol-5-yl)methyl 3-methyl-2-(methylamino)pentanoate;
55. (2R,3S)-(1-((2′-(2H-tetrazol-5-yl)biphenyl-4-yl)methyl)-2-butyl-4-chloro-1H-imidazol-5-yl)methyl 3-methyl-2-(methylamino)pentanoate;
56. (2S,3S)-(1-((2′-(2H-tetrazol-5-yl)biphenyl-4-yl)methyl)-2-butyl-4-chloro-1H-imidazol-5-yl)methyl 2-(dimethylamino)-3-methyl-pentanoate;
57. (2R,3S)-(1-((2′-(2H-tetrazol-5-yl)biphenyl-4-yl)methyl)-2-butyl-4-chloro-1H-imidazol-5-yl)methyl 2-(dimethylamino)-3-methyl-pentanoate;
58. (S)-(1-((2′-(2H-tetrazol-5-yl)biphenyl-4-yl)methyl)-2-butyl-4-chloro-1H-imidazol-5-yl)methyl 6-amino-2-(methylamino)hexanoate;
59. (R)-(1-((2′-(2H-tetrazol-5-yl)biphenyl-4-yl)methyl)-2-butyl-4-chloro-1H-imidazol-5-yl)methyl 6-amino-2-(methylamino)hexanoate;
60. (S)-(1-((2′-(2H-tetrazol-5-yl)biphenyl-4-yl)methyl)-2-butyl-4-chloro-1H-imidazol-5-yl)methyl 6-amino-2-(dimethylamino)hexanoate;
61. (R)-(1-((2′-(2H-tetrazol-5-yl)biphenyl-4-yl)methyl)-2-butyl-4-chloro-1H-imidazol-5-yl)methyl 6-amino-2-(dimethylamino)hexanoate;
62. (S)-(1-((2′-(2H-tetrazol-5-yl)biphenyl-4-yl)methyl)-2-butyl-4-chloro-1H-imidazol-5-yl)methyl 2-(methylamino)-4-(methylthio)butanoate;
63. (R)-(1-((2′-(2H-tetrazol-5-yl)biphenyl-4-yl)methyl)-2-butyl-4-chloro-1H-imidazol-5-yl)methyl 2-(methylamino)-4-(methylthio)butanoate;
64. (S)-(1-((2′-(2H-tetrazol-5-yl)biphenyl-4-yl)methyl)-2-butyl-4-chloro-1H-imidazol-5-yl)methyl 2-(dimethylamino)-4-(methylthio)butanoate;
65. (R)-(1-((2′-(2H-tetrazol-5-yl)biphenyl-4-yl)methyl)-2-butyl-4-chloro-1H-imidazol-5-yl)methyl 2-(dimethylamino)-4-(methylthio)butanoate;
66. (S)-(1-((2′-(2H-tetrazol-5-yl)biphenyl-4-yl)methyl)-2-butyl-4-chloro-1H-imidazol-5-yl)methyl 2-(methylamino)-3-phenylpropanoate;
67. (R)-(1-((2′-(2H-tetrazol-5-yl)biphenyl-4-yl)methyl)-2-butyl-4-chloro-1H-imidazol-5-yl)methyl 2-(methylamino)-3-phenylpropanoate;
68. (S)-(1-((2′-(2H-tetrazol-5-yl)biphenyl-4-yl)methyl)-2-butyl-4-chloro-1H-imidazol-5-yl)methyl 2-(dimethylamino)-3-phenylpropanoate;
69. (R)-(1-((2′-(2H-tetrazol-5-yl)biphenyl-4-yl)methyl)-2-butyl-4-chloro-1H-imidazol-5-yl)methyl 2-(dimethylamino)-3-phenylpropanoate;
70. (S)-(1-((2′-(2H-tetrazol-5-yl)biphenyl-4-yl)methyl)-2-butyl-4-chloro-1H-imidazol-5-yl)methyl 1-methylpyrrolidine-2-carboxylate;
71. (R)-(1-((2′-(2H-tetrazol-5-yl)biphenyl-4-yl)methyl)-2-butyl-4-chloro-1H-imidazol-5-yl)methyl 1-methylpyrrolidine-2-carboxylate;
72. (S)-(1-((2′-(2H-tetrazol-5-yl)biphenyl-4-yl)methyl)-2-butyl-4-chloro-1H-imidazol-5-yl)methyl 3-hydroxy-2-(methylamino)propanoate;
73. (R)-(1-((2′-(2H-tetrazol-5-yl)biphenyl-4-yl)methyl)-2-butyl-4-chloro-1H-imidazol-5-yl)methyl 3-hydroxy-2-(methylamino)propanoate;
74. (S)-(1-((2′-(2H-tetrazol-5-yl)biphenyl-4-yl)methyl)-2-butyl-4-chloro-1H-imidazol-5-yl)methyl 2-(dimethylamino)-3-hydroxypropanoate;
75. (R)-(1-((2′-(2H-tetrazol-5-yl)biphenyl-4-yl)methyl)-2-butyl-4-chloro-1H-imidazol-5-yl)methyl 2-(dimethylamino)-3-hydroxypropanoate;
76. (S)-(1-((2′-(2H-tetrazol-5-yl)biphenyl-4-yl)methyl)-2-butyl-4-chloro-1H-imidazol-5-yl)methyl 3-hydroxy-2-(methylamino)butanoate;
77. (R)-(1-((2′-(2H-tetrazol-5-yl)biphenyl-4-yl)methyl)-2-butyl-4-chloro-1H-imidazol-5-yl)methyl 3-hydroxy-2-(methylamino)butanoate;
78. (S)-(1-((2′-(2H-tetrazol-5-yl)biphenyl-4-yl)methyl)-2-butyl-4-chloro-1H-imidazol-5-yl)methyl 2-(dimethylamino)-3-hydroxybutanoate;
79. (R)-(1-((2′-(2H-tetrazol-5-yl)biphenyl-4-yl)methyl)-2-butyl-4-chloro-1H-imidazol-5-yl)methyl 2-(dimethylamino)-3-hydroxybutanoate;
80. (S)-(1-((2′-(2H-tetrazol-5-yl)biphenyl-4-yl)methyl)-2-butyl-4-chloro-1H-imidazol-5-yl)methyl 3-(4-hydroxyphenyl)-2-(methylamino)propanoate;
81. (R)-(1-((2′-(2H-tetrazol-5-yl)biphenyl-4-yl)methyl)-2-butyl-4-chloro-1H-imidazol-5-yl)methyl 3-(4-hydroxyphenyl)-2-(methylamino)propanoate;
82. (S)-(1-((2′-(2H-tetrazol-5-yl)biphenyl-4-yl)methyl)-2-butyl-4-chloro-1H-imidazol-5-yl)methyl 2-(dimethylamino)-3-(4-hydroxyphenyl)propanoate;
83. (R)-(1-((2′-(2H-tetrazol-5-yl)biphenyl-4-yl)methyl)-2-butyl-4-chloro-1H-imidazol-5-yl)methyl 2-(dimethylamino)-3-(4-hydroxyphenyl)propanoate;
84. (S)-(1-((2′-(2H-tetrazol-5-yl)biphenyl-4-yl)methyl)-2-butyl-4-chloro-1H-imidazol-5-yl)methyl 3-methyl-2-(methylamino)butanoate;
85. (R)-(1-((2′-(2H-tetrazol-5-yl)biphenyl-4-yl)methyl)-2-butyl-4-chloro-1H-imidazol-5-yl)methyl 3-methyl-2-(methylamino)butanoate;
86. (S)-(1-((2′-(2H-tetrazol-5-yl)biphenyl-4-yl)methyl)-2-butyl-4-chloro-1H-imidazol-5-yl)methyl 2-(dimethylamino)-3-methylbutanoate;
87. (R)-(1-((2′-(2H-tetrazol-5-yl)biphenyl-4-yl)methyl)-2-butyl-4-chloro-1H-imidazol-5-yl)methyl 2-(dimethylamino)-3-methylbutanoate;
88. (S)-(1-((2′-(2H-tetrazol-5-yl)biphenyl-4-yl)methyl)-2-butyl-4-chloro-1H-imidazol-5-yl)methyl 3-(1H-indol-3-yl)-2-(methylamino)propanoate;
89. (R)-(1-((2′-(2H-tetrazol-5-yl)biphenyl-4-yl)methyl)-2-butyl-4-chloro-1H-imidazol-5-yl)methyl 3-(1H-indol-3-yl)-2-(methylamino)propanoate;
90. (S)-(1-((2′-(2H-tetrazol-5-yl)biphenyl-4-yl)methyl)-2-butyl-4-chloro-1H-imidazol-5-yl)methyl 2-(dimethylamino)-3-(1H-indol-3-yl)propanoate;
91. (R)-(1-((2′-(2H-tetrazol-5-yl)biphenyl-4-yl)methyl)-2-butyl-4-chloro-1H-imidazol-5-yl)methyl 2-(dimethylamino)-3-(1H-indol-3-yl)propanoate;
92. (S)-(1-((2′-(2H-tetrazol-5-yl)biphenyl-4-yl)methyl)-2-butyl-4-chloro-1H-imidazol-5-yl)methyl 2-amino-3-phenylpropanoate;
93. (R)-(1-((2′-(2H-tetrazol-5-yl)biphenyl-4-yl)methyl)-2-butyl-4-chloro-1H-imidazol-5-yl)methyl 2-amino-3-phenylpropanoate;
94. (S)-(1-((2′-(2H-tetrazol-5-yl)biphenyl-4-yl)methyl)-2-butyl-4-chloro-1H-imidazol-5-yl)methyl 2-aminopropanoate;
95. (R)-(1-((2′-(2H-tetrazol-5-yl)biphenyl-4-yl)methyl)-2-butyl-4-chloro-1H-imidazol-5-yl)methyl 2-aminopropanoate;
96. (S)-(1-((2′-(2H-tetrazol-5-yl)biphenyl-4-yl)methyl)-2-butyl-4-chloro-1H-imidazol-5-yl)methyl 2-amino-3-hydroxypropanoate;
97. (R)-(1-((2′-(2H-tetrazol-5-yl)biphenyl-4-yl)methyl)-2-butyl-4-chloro-1H-imidazol-5-yl)methyl 2-amino-3-hydroxypropanoate;
98. (2S,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
99. (2R,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
100. (S)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
101. (R)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
102. (S)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
103. (R)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
104. (S)-(1-((2′-(2H-tetrazol-5-yl)biphenyl-4-yl)methyl)-2-butyl-4-chloro-1H-imidazol-5-yl)methyl 2-amino-3-methylbutanoate;
105. (R)-(1-((2′-(2H-tetrazol-5-yl)biphenyl-4-yl)methyl)-2-butyl-4-chloro-1H-imidazol-5-yl)methyl 2-amino-3-methylbutnoate;
106. (2S,3R)-(1-((2′-(2H-tetrazol-5-yl)biphenyl-4-yl)methyl)-2-butyl-4-chloro-1H-imidazol-5-yl)methyl 2-amino-3-methylpentanoate;
107. (2R,3R)-(1-((2′-(2H-tetrazol-5-yl)biphenyl-4-yl)methyl)-2-butyl-4-chloro-1H-imidazol-5-yl)methyl 2-amino-3-methylpentanoate;
108. (S)-(1-((2′-(2H-tetrazol-5-yl)biphenyl-4-yl)methyl)-2-butyl-4-chloro-1H-imidazol-5-yl)methyl 2,5-diamino-5-oxopentanoate;
109. (R)-(1-((2′-(2H-tetrazol-5-yl)biphenyl-4-yl)methyl)-2-butyl-4-chloro-1H-imidazol-5-yl)methyl 2,5-diamino-5-oxopentanoate;
110. (S)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
111. (R)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
112. (S)-(1-((2′-(2H-tetrazol-5-yl)biphenyl-4-yl)methyl)-2-butyl-4-chloro-1H-imidazol-5-yl)methyl 2,6-diaminohexanoate;
113. (R)-(1-((2′-(2H-tetrazol-5-yl)biphenyl-4-yl)methyl)-2-butyl-4-chloro-1H-imidazol-5-yl)methyl 2,6-diaminohexanoate;
114. (S)-(1-((2′-(2H-tetrazol-5-yl)biphenyl-4-yl)methyl)-2-butyl-4-chloro-1H-imidazol-5-yl)methyl 2-amino-4-(methylthio)butanoate;
115. (R)-(1-((2′-(2H-tetrazol-5-yl)biphenyl-4-yl)methyl)-2-butyl-4-chloro-1H-imidazol-5-yl)methyl 2-amino-4-(methylthio)butanoate;
116. (S)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
117. (R)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
118. (S)-(1-((2′-(2H-tetrazol-5-yl)biphenyl-4-yl)methyl)-2-butyl-4-chloro-1H-imidazol-5-yl)methyl 2-aminoacetate;
119. (R)-(1-((2′-(2H-tetrazol-5-yl)biphenyl-4-yl)methyl)-2-butyl-4-chloro-1H-imidazol-5-yl)methyl 2-aminoacetate;
120. (S)-(1-((2′-(2H-tetrazol-5-yl)biphenyl-4-yl)methyl)-2-butyl-4-chloro-1H-imidazol-5-yl)methyl 2-amino-5-guanidinopentanoate;
121. (R)-(1-((2′-(2H-tetrazol-5-yl)biphenyl-4-yl)methyl)-2-butyl-4-chloro-1H-imidazol-5-yl)methyl 2-amino-5-guanidinopentanoate;
122. (S)-4-((1-((2′-(2H-tetrazol-5-yl)biphenyl-4-yl)methyl)-2-butyl-4-chloro-1H-imidazol-5-yl)methoxy)-3-amino-4-oxobutanoic acid;
123. (R)-4-((1-((2′-(2H-tetrazol-5-yl)biphenyl-4-yl)methyl)-2-butyl-4-chloro-1H-imidazol-5-yl)methoxy)-3-amino-4-oxobutanoic acid;
124. (S)-(1-((2′-(2H-tetrazol-5-yl)biphenyl-4-yl)methyl)-2-butyl-4-chloro-1H-imidazol-5-yl)methyl 2,4-diamino-4-oxobutanoate;
125. (R)-(1-((2′-(2H-tetrazol-5-yl)biphenyl-4-yl)methyl)-2-butyl-4-chloro-1H-imidazol-5-yl)methyl 2,4-diamino-4-oxobutanoate;
126. (S)-(1-((2′-(2H-tetrazol-5-yl)biphenyl-4-yl)methyl)-2-butyl-4-chloro-1H-imidazol-5-yl)methyl 2-amino-3-mercaptopropanoate;
127. (R)-(1-((2′-(2H-tetrazol-5-yl)biphenyl-4-yl)methyl)-2-butyl-4-chloro-1H-imidazol-5-yl)methyl 2-amino-3-mercaptopropanoate;
128. 1-((2′-(2H-tetrazol-5-yl)biphenyl-4-yl)methyl)-2-butyl-4-chloro-1H-imidazol-5-yl)methyl 2-methyl-2-(methylamino)propanoate;
129. 1-((2′-(2H-tetrazol-5-yl)biphenyl-4-yl)methyl)-2-butyl-4-chloro-1H-imidazol-5-yl)methyl 2-(dimethylamino)-2-methylpropanoate;
130. 1-((2′-(2H-tetrazol-5-yl)biphenyl-4-yl)methyl)-2-butyl-4-chloro-1H-imidazol-5-yl)methyl 1-(methylamino)cyclopropanecarboxylate;
131. 1-((2′-(2H-tetrazol-5-yl)biphenyl-4-yl)methyl)-2-butyl-4-chloro-1H-imidazol-5-yl)methyl 1-(dimethylamino)cyclopropanecarboxylate;
132. 1-((2′-(2H-tetrazol-5-yl)biphenyl-4-yl)methyl)-2-butyl-4-chloro-1H-imidazol-5-yl)methyl 1-(methylamino)cyclobutanecarboxylate;
133. 1-((2′-(2H-tetrazol-5-yl)biphenyl-4-yl)methyl)-2-butyl-4-chloro-1H-imidazol-5-yl)methyl 1-(dimethylamino)cyclobutanecarboxylate;
134. (S)-(1-((2′-(2H-tetrazol-5-yl)biphenyl-4-yl)methyl)-2-butyl-4-chloro-1H-imidazol-5-yl)methyl 2-amino-4-methylpentanoate; and
135. (R)-(1-((2′-(2H-tetrazol-5-yl)biphenyl-4-yl)methyl)-2-butyl-4-chloro-1H-imidazol-5-yl)methyl 2-amino-4-methylpentanoate;

In another embodiment, the present invention is related to a compound selected from a group consisting of:
(S)-(1-((2′-(2H-tetrazol-5-yl)biphenyl-4-yl)methyl)-2-butyl-4-chloro-1H-imidazol-5-yl)methyl 2-aminopropanoate;
(S)-(1-((2′-(2H-tetrazol-5-yl)biphenyl-4-yl)methyl)-2-butyl-4-chloro-1H-imidazol-5-yl)methyl pyrroldine-2-carboxylate;
(S)-(1-((2′-(2H-tetrazol-5-yl)biphenyl-4-yl)methyl)-2-butyl-4-chloro-1H-imidazol-5-yl)methyl 2-amino-3-phenylpropanoate;
(S)-(1-((2′-(2H-tetrazol-5-yl)biphenyl-4-yl)methyl)-2-butyl-4-chloro-1H-imidazol-5-yl)methyl 2-amino-3-hydroxypropanoate;
(2S,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
(S)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
(S)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
(S)-(1-((2′-(2H-tetrazol-5-yl)biphenyl-4-yl)methyl)-2-butyl-4-chloro-1H-imidazol-5-yl)methyl 2-amino-3-methylbutanoate;
(2S,3R)-(1-((2′-(2H-tetrazol-5-yl)biphenyl-4-yl)methyl)-2-butyl-4-chloro-1H-imidazol-5-yl)methyl 2-amino-3-methylpentanoate;
(S)-(1-((2′-(2H-tetrazol-5-yl)biphenyl-4-yl)methyl)-2-butyl-4-chloro-1H-imidazol-5-yl)methyl 2,5-diamino-5-oxopentanoate;
(S)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
(S)-(1-((2′-(2H-tetrazol-5-yl)biphenyl-4-yl)methyl)-2-butyl-4-chloro-1H-imidazol-5-yl)methyl 2,6-diaminohexanoate;
(S)-(1-((2′-(2H-tetrazol-5-yl)biphenyl-4-yl)methyl)-2-butyl-4-chloro-1H-imidazol-5-yl)methyl 2-amino-4-(methylthio)butanoate;
(S)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
(S)-(1-((2′-(2H-tetrazol-5-yl)biphenyl-4-yl)methyl)-2-butyl-4-chloro-1H-imidazol-5-yl)methyl 2-aminoacetate;
(S)-(1-((2′-(2H-tetrazol-5-yl)biphenyl-4-yl)methyl)-2-butyl-4-chloro-1H-imidazol-5-yl)methyl 2-amino-5-guanidinopentanoate;
(S)-(1-((2′-(2H-tetrazol-5-yl)biphenyl-4-yl)methyl)-2-butyl-4-chloro-1H-imidazol-5-yl)methyl 2,4-diamino-4-oxobutanoate; and
(S)-5-((1-((2′-(2H-tetrazol-5-yl)biphenyl-4-yl)methyl)-2-butyl-4-chloro-1H-imidazol-5-yl)methoxy)-3-amino-4-oxobutanoic acid.

The invention may be embodied in other specific forms without departing from the spirit or essential attributes thereof. This invention also encompasses all combinations of alternative aspects of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All chiral, diastereomeric, racemic forms and all geometric isomeric forms of a structure are intended, unless the specific stereochemistry or isomeric form is specifically indicated. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced.

When any variable (e.g., R₂) occurs more than one time in any constituent or formula for a compound, its definition at each occurrence is independent of its definition at every other occurrence. Thus, for example, if a group is shown to be substituted with 0-2 R₂, then said group may optionally be substituted with up to two R₂ groups and R₂ at each occurrence is selected independently from the definition of R₂. Also, combinations of substituents and/or variables are permissible if such combinations result in stable compounds.

As used herein, “C₁₋₆ alkyl” is intended to include both branched and straight-chain saturated aliphatic hydrocarbon groups having the specified number of carbon atoms, examples of which include, but are not limited to, methyl, ethyl, n-propyl, i-propyl, n-butyl, i-butyl, sec-butyl, t-butyl, pentyl, and hexyl. C₁₋₆ alkyl, is intended to include C₁, C₂, C₃, C₄, C₅, C₆, alkyl groups. C₁₋₈ alkyl is intended to include C₁, C₂, C₃, C₄, C₅, C₆, C₇, C₈, alkyl groups. “Alkenyl” is intended to include hydrocarbon chains of either a straight or branched configuration and one or more unsaturated carbon-carbon bonds which may occur in any stable point along the chain, such as ethenyl, propenyl, and the like. “C₃₋₆ cycloalkyl” is intended to include saturated ring groups having the specified number of carbon atoms in the ring, including mono-, bi-, or poly-cyclic ring systems, such as cyclopropyl, cyclobutyl, cyclopentyl, cyclohexyl, and C₃₋₆ cycloalkyl, is intended to include C₃, C₄, C₅, and C₆ cycloalkyl groups.

As used herein, the term “amine protecting group” (or “N-protected”) refers to any group known in the art of organic synthesis for the protection of amine groups. As used herein, the term “amine protecting group reagent” refers to any reagent known in the art of organic synthesis for the protection of amine groups which may be reacted with an amine to provide an amine protected with an amine protecting group. The “amine protecting group” should be compatible with other reaction conditions. Such amine protecting groups include those listed in T. W. Greene, “Protective Groups in Organic Synthesis” 4^{th} edition, John Wiley & Sons, New York (2006) and “The Peptides: Analysis, Synthesis, Biology”, Vol. 3, Academic Press, New York (1981), and the disclosures of which are hereby incorporated by reference in their entirety. Examples of amine protecting groups include, but are not limited to, the following: 1) acyl types such as formyl, trifluoroacetyl, and p-toluenesulfonyl; 2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; and 4) cyclic alkyl carbamate types such as cyclopentyloxycarbonyl and adamantyloxycarbonyl.

As used herein, “carbocycle” or “carbocyclic residue” is intended to mean any stable 3, 4, 5, 6, or 7-membered monocyclic, any of which may be saturated, partially unsaturated, or aromatic. Examples of such carbocycles include, but are not limited to, cyclopropyl, cyclobutyl, cyclopentyl, cyclohexyl.

As used herein, the term “heterocycle” or “heterocyclic system” is intended to mean a stable 5, 6, or 7-membered monocyclic or bicyclic or heterocyclic ring which is saturated, partially unsaturated or unsaturated (aromatic), and which consists of carbon atoms and 1, 2, 3, or 4 heteroatoms independently selected from the group consisting of N, NH, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. As used herein, the term “aromatic heterocyclic system” is intended to mean a stable 5- to 7-membered monocyclic or bicyclic or heterocyclic aromatic ring which consists of carbon atoms and from 1 to 4 heteroatoms independently selected from the group consisting of N, O and S.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, chromanyl, chromenyl, cinnolinyl, decahydroquinolinyl, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, tetrazolyl, and xanthenyl. Preferred heterocycles include, but are not limited to, pyridinyl, thiophenyl, furanyl, indazolyl, benzothiazolyl, benzimidazolyl, benzothiaphenyl, benzofuranyl, benzoxazolyl, benzisoxazolyl, quinolinyl, isoquinolinyl, imidazolyl, indolyl, isoidolyl, piperidinyl, piperidonyl, 4-piperidonyl, piperonyl, pyrrazolyl, 1,2,4-triazolyl, 1,2,3-triazolyl, tetrazolyl, thiazolyl, oxazolyl, pyrazinyl, and pyrimidinyl. Also included are fused ring and spiro compounds containing, for example, the above heterocycles.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds may form salts which are also within the scope of this invention. Reference to a compound of the formula (I) herein is understood to include reference to salts thereof, unless otherwise indicated. The term “salt(s),” as employed herein, denotes acidic and/or basic salts formed with inorganic and/or organic acids and bases. In addition, when a compound of formula (I) contains both a basic moiety, such as, but not limited to an amine or a pyridine ring, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula (I) may be formed, for example, by reacting a compound of the formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, and the disclosure of which is hereby incorporated by reference in its entirety.

The compounds disclosed herein, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention. Additionally, inventive compounds may have trans and cis isomers and may contain one or more chiral centers, therefore existing in enantiomeric and diastereomeric forms. The invention includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form.

“Stable compound” and “stable structure” are meant to indicate a compound that is sufficiently robust to survive isolation to a useful degree of purity from a reaction mixture, and formulation into an efficacious therapeutic agent. Stable compounds are envisioned within the scope of this invention.

“Therapeutically effective amount” is intended to include an amount of a compound of the present invention alone or an amount of the combination of compounds claimed or an amount of a compound of the present invention in combination with other active ingredients effective to treat the circulatory diseases described herein.

“Substantially pure” as used herein is intended to include a compound having a purity greater than about 90 weight percent, including 90, 91, 92, 93, 94, 95, 96, 97, 98, 99, and 100 percent.

“Base” includes both organic and inorganic bases. The strength of the individual base will depend on the specific reaction. Examples of bases include, but are not limited to, TEA (triethylamine), NMM (N-methyl morpholine), pyridine, NaH, NaOBu-t, KOBu-t, ethyldiisopropylamine, NaOH, KOH and/or LiOH.

“Suitable solvent” as used herein is intended to refer to a single solvent as well as mixtures of solvents. Solvents may be selected, as appropriate for a given reaction step, from, for example, aprotic polar solvents such as DMF, DMA, DMSO, dimethylpropyleneurea, N-methylpyrrolidone (NMP), and hexamethylphosphoric triamide; ether solvents such as diethyl ether, THF, 1,4-dioxane, methyl t-butyl ether, dimethoxymethane, and ethylene glycol dimethyl ether; alcohol solvents such as MeOH, EtOH, and isopropanol; and halogen-containing solvents such as methylene chloride, chloroform, carbon tetrachloride, and 1,2-dichloroethane. Mixtures of solvents may also include biphasic mixtures.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The abbreviations in Table 1 may be used herein and/or in the Examples.

TABLE 1
Abbreviations.

| | |
|---|---|
| DCC = 1,3-dicyclohexylcarbodiimide | EDC (or EDC.HCl) or EDCl (or EDCl.HCl) |
| CDI = carbonyl diimidazole | or EDAC = 3-ethyl-3-(dimethylamino)propyl- |
| Ph = phenyl | carbodiimide hydrochloride (or 1-(3- |
| Bn = benzyl | dimethylaminopropyl)-3-ethylcarbodiimide |
| t-Bu = tertiary butyl | hydrochloride) |
| Me = methyl | HOBT or HOBT•H₂O = 1- |
| Et = ethyl | hydroxybenzotriazole hydrate |
| Boc = tert-butoxycarbonyl | HOAT = 1-Hydroxy-7-azabenzotriazole |
| Cbz = carbobenzyloxy or carbobenzoxy or | BOP reagent = benzotriazol-1-yloxy-tris |
| benzyloxycarbonyl | (dimethylamino) phosphonium |
| THF = tetrahydrofuran | hexafluorophosphate |
| Et₂O = diethyl ether | DEAD = diethyl azodicarboxylate |
| hex = hexanes | DIAD = diisopropyl azodicarboxylate |
| EtOAc = ethyl acetate | Cbz-Cl = benzyl chloroformate |
| DMF = dimethyl formamide | N₂ = nitrogen |
| MeOH = methanol | min = minute(s) |
| EtOH = ethanol | h or hr = hour(s) |
| i-PrOH = isopropanol | L = liter |
| DMSO = dimethyl sulfoxide | mL = milliliter |
| DME = 1,2 dimethoxyethane | mᵤL = microliter |
| DCE = 1,2 dichloroethane | g = gram(s) |
| HOAc or AcOH = acetic acid | mg = milligram(s) |
| TFA = trifluoroacetic acid [ | mol = moles |
| i-Pr₂NEt = diisopropylethylamine | mmol = millimole |
| Et₃N = triethylamine | uM = micromolar |
| NMM = N-methyl morpholine | RT = room temperature |
| DMAP = 4-dimethylaminopyridine | sat or sat'd = saturated |
| Pd/C = palladium on carbon | aq. = aqueous |
| KOH = potassium hydroxide | TLC = thin layer chromatography |
| NaOH = sodium hydroxide | HPLC = high performance liquid |
| LiOH = lithium hydroxide | chromatography |
| K₂CO₃ = potassium carbonate | LC/MS = high performance liquid |
| NaHCO₃ = sodium bicarbonate | chromatography/mass spectrometry |
| DBU = 1,8-diazabicyclo[5.4.0]undec-7-ene | MS or Mass Spec = mass spectrometry |
| | NMR = nuclear magnetic resonance |
| | NMR spectral data: s = singlet; d = doublet; |
| | m = multiplet; br = broad; t = triplet; |
| | mp = melting point |

Synthesis. A compound of formula (I) can be produced by, for example, a method shown in the following or a method analogous thereto and the like. The reactions are performed in a solvent appropriate to the reagents and materials employed and suitable for the transformations being effected. It will be understood by those skilled in the art of organic synthesis that the functionality present on the molecule should be consistent with the transformations proposed. This may sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Protective Groups In Organic Synthesis, The third edition by Greene and Wuts (Wiley and Sons, 2006), the disclosure of which is hereby incorporated by reference in its entirety.

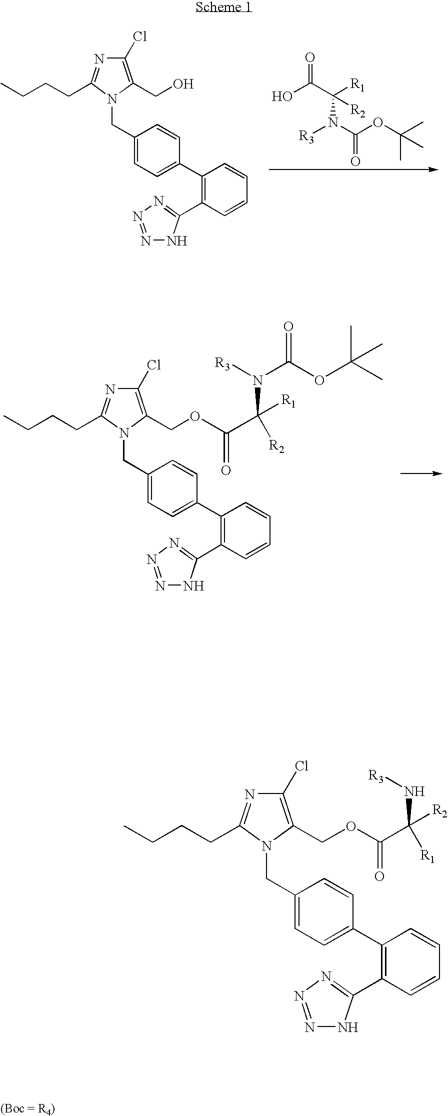

Compounds of this invention could be synthesized using the procedures summarized in Scheme 1. The commercially available (1-((2′-(2H-tetrazol-5-yl)biphenyl-4-yl)methyl)-2-butyl-4-chloro-1H-imidazol-5-yl)methanol is condensed with the acid in suitable solvents under esterification condition (for example, via a mixed acid anhydride, an acid halide, EDC and the like) to form ester derivative of (1-((2′-(2H-tetrazol-5-yl)biphenyl-4-yl)methyl)-2-butyl-4-chloro-1H-imidazol-5-yl)methanol. If needed, the product may further be deprotected to free base and the free base is reacted with acid to form to salt form of compounds of this invention.

While the yield of compounds of formula (I) may vary depending on the reaction conditions used, compound (I) can be obtained easily at a high purity by a conventional means of separation or purification (e.g., recrystallization, column chromatography and the like) from the product by such methods.

When compound (I) is obtained as a free form, it can be converted to an object salt by a method known per se or a method analogous thereto. Conversely, when it is obtained as a salt, it can be converted to a free form or a different object salt by a method known per se or a method analogous thereto. Compounds of formula (I) are preferably salt forms.

When optical isomers of compounds of formula (I) exist, such individual optical isomers and a mixture thereof are all naturally encompassed in the scope of the present invention.

Compounds of formula (I) may be a crystal, and may have a form of a single crystal or a form of a mixture of plural crystals. Crystals can be produced by crystallization according to a crystallization method known per se.

Compounds of formula (I) may be a solvate (e.g., hydrate etc.) and both solvate and non-solvate (e.g., non-hydrate etc.) are encompassed in the scope of the present invention.

Use of Compounds of Formula (I). The compound of the present invention thus produced shows lower toxicity and is safe (in other words, more superior as a pharmaceutical agent from the aspects of acute toxicity, chronic toxicity, genetic toxicity, reproductive toxicity, cardiac toxicity, drug interaction, carcinogenicity and the like), and rapidly converted to compound (II) in the living body of an animal, particularly a mammal (e.g., human, monkey, cat, pig, horse, bovine, mouse, rat, guinea pig, dog, rabbit etc.).

Since in the living body, compounds of Formula (I) can be rapidly converted to compound (II), which has a strong angiotensin II antagonistic activity, the compound of the present invention is useful as an agent for the prophylaxis or treatment of a disease (or a disease whose onset is promoted) developed by the contraction or growth of blood vessels or organ disorder, which expresses via an angiotensin II receptor, or due to the presence of angiotensin II, or a factor induced by the presence of angiotensin II, in mammals (e.g., human, monkey, cat, pig, horse, bovine, mouse, rat, guinea pig, dog, rabbit etc.).

As such diseases, for example, hypertension, blood pressure circadian rhythm abnormality, heart diseases (e.g., cardiac hypertrophy, acute heart failure and chronic heart failure including congestive heart failure, cardiac myopathy, angina pectoris, myocarditis, atrial fibrillation, arrhythmia, tachycardia, cardiac infraction etc.), cerebrovascular disorders (e.g., asymptomatic cerebrovascular disorder, transient cerebral ischemia, apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after cardiac infarction, renal diseases (e.g., nephritis, glomerulonephritis, glomerulosclerosis, renal failure, thrombotic vasculopathy, complication of dialysis, organ dysfunction including nephropathy by radiation damage etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or obliteration and organ disorders after intervention (e.g., percutaneous transluminal coronary angioplasty, stenting, coronary angioscopy, intravascular ultrasound, dounce thrombolytic therapy etc.), vascular re-obliteration and restenosis after bypass, polycythemia, hypertension, organ disorder and vascular hypertrophy after transplantation, rejection after transplantation, ocular diseases (e.g., glaucoma, ocular hypertension etc.), thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, obstructive thromboangiitis, ischemic cerebral circulatory disorder, Raynaud's disease, Berger disease etc.).

Since the compound of the present invention can maintain a constant hypotensive action both day and night, reduction of the dose and frequency is possible as compared to the administration of compound II. In addition, it can effectively suppress particularly problematic increase in the blood pressure before and after rising in patients with hypertension.

The compound of the present invention is useful as an agent for the prophylaxis or treatment of metabolic syndrome. Because patients with metabolic syndrome have an extreme high incidence of cardiovascular diseases as compared to patients with single lifestyle-related diseases, the prophylaxis or treatment of metabolic syndrome is quite important to prevent cardiovascular diseases.

The compound of the present invention can be used for treating patients of high blood pressure with metabolic syndrome.

The compound of the present invention can be used as a safe pharmaceutical agent to mammals (e.g., human, monkey, cat, swine, horse, bovine, mouse, rat, guinea pig, dog, rabbit and the like) in the form of the compound as it is or a pharmaceutical composition after mixing with a pharmacologically acceptable carrier according to a method known per se.

As used herein, as the pharmacologically acceptable carrier, various organic or inorganic carrier substances conventionally used as materials for preparations can be used. For example, excipient, lubricant, binder and disintegrant for solid preparations; solvent, dissolution aids, suspending agent, isotonizing agent and buffer for liquid preparations; and the like can be mentioned. Where necessary, additives for preparation, such as preservative, antioxidant, coloring agent, sweetening agent and the like, can be also used.

Although typically any excipient may be used, preferable examples of excipient include lactose, sucrose, D-mannitol, D-sorbitol, starch, pregelatinized starch, dextrin, crystalline cellulose, low-substituted hydroxypropyl cellulose, carboxymethyl cellulose sodium, gum arabic, pullulan, light silicic anhydride, synthetic aluminum silicate, magnesium aluminometasilicate and the like.

Although typically any lubricant may be used, preferable examples of lubricant include magnesium stearate, calcium stearate, colloidal silica and the like.

Although typically any binder may be used, preferable examples of binder include pregelatinized starch, sucrose, gelatin, gum arabic, methyl cellulose, carboxymethyl cellulose, carboxymethyl cellulose sodium, crystalline cellulose, sucrose, D-mannitol, trehalose, dextrin, pullulan, hydroxypropyl cellulose, hydroxypropylmethyl cellulose, polyvinylpyrrolidone and the like.

Although any disintegrant typically may be used, preferable examples of disintegrant include lactose, sucrose, starch, carboxymethyl cellulose, carboxymethyl cellulose calcium, croscarmellose sodium, carboxymethyl starch sodium, light silicic anhydride, low-substituted hydroxypropyl cellulose and the like.

Although typically any solvent may be used, preferable examples include water for injection, physiological brine, Ringer's solution, alcohol, propylene glycol, polyethylene glycol, sesame oil, corn oil, olive oil, cottonseed oil and the like.

Although typically any dissolution aid may be used, preferable examples of dissolution aids include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Although typically any suspending agent may be used, preferable examples of suspending agent include surfactants such as stearyltriethanolamine, sodium lauryl sulfate, lauryl aminopropionate, lecithin, benzalkonium chloride, benzethonium chloride, glycerol monostearate etc.; hydrophilic polymers such as polyvinyl alcohol, polyvinylpyrrolidone, carboxymethylcellulose sodium, ethylcellulose, hydroxymethylcellulose, hydroxyethylcellulose, hydroxypropylcellulose etc.; polysorbates, polyoxyethylene hydrogenated castor oil and the like.

Although typically any isotonizing agent may be used, preferable examples include sodium chloride, glycerin, D-mannitol, D-sorbitol, glucose and the like.

Although typically any buffer may be used, preferable examples of buffer include buffers such as phosphate, acetate, carbonate, citrate etc., and the like.

Although typically any preservative may be used, preferable examples of preservative include p-oxybenzoate, chlorobutanol, benzyl alcohol, phenethyl alcohol, dehydroacetic acid, sorbic acid and the like.

Although any antioxidant may be used, typically, preferable examples of antioxidant include sulfite, ascorbate and the like.

Although any coloring agent may be used, typically, preferable examples include water-soluble edible tar dyes (e.g., food colors such as Food Red Nos. 2 and 3, Food Yellow Nos. 4 and 5, Food Blue Nos. 1 and 2 etc.), water-insoluble Lake dyes (e.g., aluminum salts of the aforementioned water-soluble edible tar dyes etc.), natural colors (e.g., .beta.-carotene, chlorophyll, iron oxide red etc.) and the like.

Although any sweetening agent may be used, typically, preferable examples of sweetening agent include saccharin sodium, dipotassium glycyrrhizinate, aspartame, stevia and the like.

The dosage form of the pharmaceutical composition includes, for example, oral agents such as tablet, capsule (including soft capsule and microcapsule), granule, powder, syrup, emulsion, suspension, sustained-release preparation and the like, which can be each safely administered orally.

The pharmaceutical composition can be prepared by conventional methods in the field of pharmaceutical manufacturing technical field, such as methods described in the US Pharmacopoeia, and the like. Specific production methods for such preparations are hereinafter described in detail.

For example, a tablet is produced by adding, for example, excipients (e.g., lactose, sucrose, starch, D-mannitol etc.), disintegrants (e.g., carboxymethyl cellulose calcium etc.), binders (e.g., pregelatinized starch, gum arabic, carboxymethyl cellulose, hydroxypropyl cellulose, polyvinylpyrrolidone etc.), lubricants (e.g., talc, magnesium stearate, polyethylene glycol 6000 etc.) and the like, to the active ingredient, compression-shaping, and, where necessary, applying a coating by a method known per se using coating base known per se for the purpose of achieving taste masking, enteric dissolution or sustained release.

The capsule can be made as a hard capsule filled with a powder or granular pharmaceutical agent, or a soft capsule filled with a liquid or suspension liquid. The hard capsule is produced by mixing and/or granulating an active ingredient with, for example, an excipient (e.g., lactose, sucrose, starch, crystalline cellulose, D-mannitol and the like), a disintegrant (low substituted hydroxypropyl cellulose, carmellose calcium, corn starch, croscarmellose sodium and the like), a binder (hydroxypropyl cellulose, polyvinylpyrrolidone, hydroxypropylmethyl cellulose and the like), a lubricant (magnesium stearate and the like) and the like, and filling the mixture or granule in a capsule formed from the aforementioned gelatin, hydroxypropylmethyl cellulose and the like. The soft capsule is produced by dissolving or suspending the active ingredient in a base (soybean oil, cottonseed oil, medium chain fatty acid triglyceride, beeswax and the like) and sealing the prepared solution or suspension in a gelatin sheet using, for example, a rotary filling machine and the like.

When a compound of formula (I) is a salt and avoidance of contact of the compound in the form of a salt with water is preferable, the compound is preferably dry-mixed with an excipient and the like to give a hard capsule.

The content of a compound of formula (I) in a pharmaceutical composition is generally about 0.01-about 99.9 wt %, preferably about 0.1-about 50 wt %, relative to the entire preparation.

The dose of compound (I) is determined in consideration of age, body weight, general health condition, sex, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for essential hypertension in adult, the daily dose of 1-100 mg is preferably administered in a single dose or in 2 or 3 portions.

In addition, because the compound of the present invention is superior in safety, it can be administered for a long period.

The compound of the present invention can be used in combination with pharmaceutical agents such as a therapeutic agent for diabetes, a therapeutic agent for diabetic complications, an anti-hyperlipidemia agent, an anti-arteriosclerotic agent, an anti-hypertensive agent, an anti-obesity agent, a diuretic, an antigout agent, an antithrombotic agent, an anti-inflammatory agent, a chemotherapeutic agent, an immunotherapeutic agent, a therapeutic agent for osteoporosis, an anti-dementia agent, an erectile dysfunction amelioration agent, a therapeutic agent for urinary incontinence/urinary frequency and the like (hereinafter to be abbreviated as a combination drug). On such occasions, the timing of administration of the compound of the present invention and that of the combination drug is not limited, as long as the compound of the present invention and the combination drug are combined. As the mode of such administration, for example, (1) administration of a single preparation obtained by simultaneous formulation of the compound of the present invention and a combination drug, (2) simultaneous administration of two kinds of preparations obtained by separate formulation of the compound of the present invention and a combination drug, by a single administration route, (3) time staggered administration of two kinds of preparations obtained by separate formulation of the compound of the present invention and a combination drug, by the same administration route, (4) simultaneous administration of two kinds of preparations obtained by separate formulation of the compound of the present invention and a combination drug, by different administration routes, (5) time staggered administration of two kinds of preparations obtained by separate formulation of the compound of the present invention and a combination drug, by different administration routes, such as administration in the order of the compound of the present invention and then the combination drug, or administration in a reversed order, and the like can be mentioned. The dose of the combination drug can be appropriately determined based on the dose clinically employed. The mixing ratio of the compound of the present invention and the combination drug can be appropriately selected according to the administration subject, administration route, target disease, condition, combination, and other factors. In cases where the administration subject is human, for example, the combination drug may be used in an amount of 0.01 to 100 parts by weight per part by weight of the compound of the present invention.

As the anti-hyperlipidemia agents, for example, statin compounds which are cholesterol synthesis inhibitors (e.g., cerivastatin, pravastatin, simvastatin, lovastatin, atorvastatin, fluvastatin, itavastatin or salts thereof (e.g., sodium salt etc.), squalene synthetase inhibitors or fibrate compounds having a triglyceride lowering effect (e.g., bezafibrate, clofibrate, simfibrate, clinofibrate etc.) and the like can be mentioned.

As the anti-arteriosclerotic agents, for example, an acyl-Coenzyme A cholesterol acyltransferase (ACAT) inhibitor (e.g. melinamide, etc.) and a lipid rich plaque regressing agent (e.g. compounds described in WO 02/06264, WO 03/059900 etc.) and the like can be mentioned.

As the antihypertensive agents, for example, angiotensin converting enzyme inhibitors (e.g., captopril, enalapril, delapril etc.), angiotensin II antagonists (e.g., candesartan cilexetil, candesartan, losartan, losartan potassium, eprosartan, valsartan, termisartan, irbesartan, tasosartan, olmesartan, olmesartan medoxomil etc.), calcium antagonists (e.g., manidipine, nifedipine, amlodipine, efonidipine, nicardipine etc.), beta-blocker (e.g., metoprolol, atenolol, propranolol, carvedilol, pindolol etc.), clonidine and the like can be mentioned.

As the antithrombotic agents, for example, anticoagulating agent [e.g., heparin sodium, heparin potassium, warfarin potassium (warfarin), activated blood coagulation factor X inhibitor (e.g., compounds described in WO 2004/048363 etc.)], thrombolytic agent [e.g., tPA, urokinase], antiplatelet agent [e.g., aspirin, sulfinpyrazone (anturan), dipyridamole (persantin), ticlopidine (panaldine), cilostazol (pletal), GPIIb/IIIa antagonist (ReoPro), clopidogrel etc.], and the like can be mentioned.

The above-mentioned combination drug may be a combination of two or more kinds thereof combined at appropriate ratios.

The compound of the present invention shows a superior prophylactic or therapeutic effect against circulatory diseases such as hypertension and the like and metabolic diseases such as diabetes and the like.

### EXAMPLES

The present invention is explained in detail by referring to the following Examples. However, these Examples are mere practical embodiments and do not limit the present invention. The present invention may be modified as long as the scope of the invention is not deviated.

The elution by column chromatography in Examples was performed under observation by TLC (thin-layer chromatography). In the TLC observation, 60F₂₅₄ (Merck) was used as a TLC plate, the solvent used as an elution solvent in the column chromatography was used as a developing solvent, and UV detector was used for detection. As silica gel for column, Kieselgel 60 (70 230 mesh) or Kieselgel 60 (230 400 mesh) manufactured by Merck was used.

### Example 1

### General Procedure

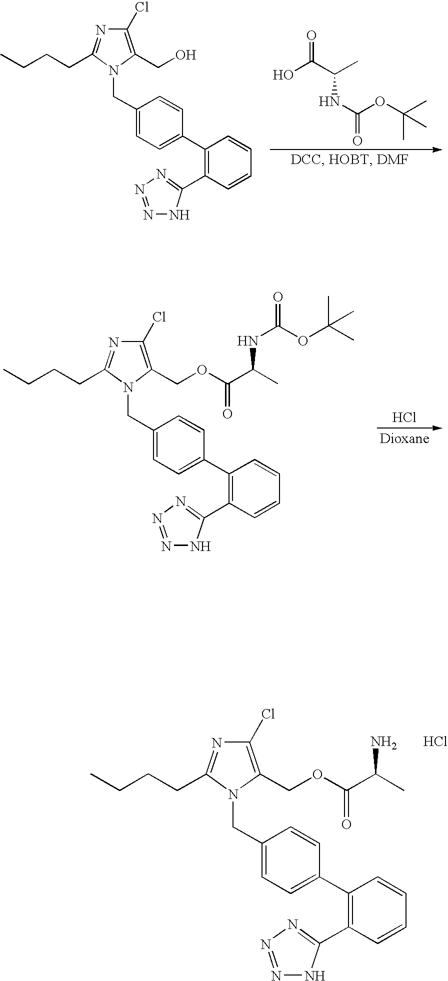

1) (S)-(1-((2′-(2H-tetrazol-5-yl)biphenyl-4-yl)methyl)-2-butyl-4-chloro-1H-imidazol-5-yl)methyl 2-(tert-butoxycarbonyl)aminopropanoate. (1-((2′-(2H-tetrazol-5-yl)biphenyl-4-yl)methyl)-2-butyl-4-chloro-1H-imidazol-5-yl)methanol (422 mg, 1.0 mmol) was dissolved in DMF (8.0 mL) at RT and then cool to 0° C. To this solution were added N-(tert-butoxycarbonyl)-L-alanine (378 mg, 2.0 mmol), EDCCl (383 mg, 2.0 mmol), HOBT (270 mg, 2.0 mmol) and N-methylmorpholine (202 mg, 2.0 mmol). The reaction was stirred at 0° C. for 30 min and at RT for 24 h. The reaction was diluted with water (150 ml) and extracted with Ethyl acetate (200 ml). The organic extract was washed by a solution of citric acid in water (10%), water and Sat'd NaCl solution, dried over MgSO₄ anhydrous and concentrated with reduced pressure to give glassy solid material. Silica gel liquid chromatography(EtAc/Hexane/AcOH v/v/v=80/100/3) of the material provided the compound, (S)-(1-((2′-(2H-tetrazol-5-yl)biphenyl-4-yl)methyl)-2-butyl-4-chloro-1H-imidazol-5-yl)methyl 2-(tert-butoxycarbonyl)aminopropanoate (470 mg, 79%) as a colorless solid. The compound is C₃₀H₃₆ClN₇O₄, LC-MS (ESI, m/z): 592.4[M−H]⁻.

2) (S)-(1-((2′-(2H-tetrazol-5-yl)biphenyl-4-yl)methyl)-2-butyl-4-chloro-1H-imidazol-5-yl)methyl 2-aminopropanoate. (S)-(1-((2′-(2H-tetrazol-5-yl)biphenyl-4-yl)methyl)-2-butyl-4-chloro-1H-imidazol-5-yl)methyl 2-(tert-butoxycarbonyl)aminopropanoate (672 mg, 1.13 mmol) was dissolved in acetonitrile (5 ml) and to this solution was added p-toluenesulfonic acid (975 mg, 5.67 mmol). This reaction was stirred at RT for 30 min and concentrated with reduced pressure. The concentrate was dissolved in isopropanol/CH₂Cl₂ (v/v=⅓, 150 ml) and adjusted pH to 9.0 with sat'd NaHCO₃ water solution. After partition, isopropanol/CH₂Cl₂ layer was separated and washed with sat'd NaCl water solution and dried over anhydrous Na₂SO₄. Then the clear solution was added citric acid water solution (10%, w/v, 0.53 ml) to form white solid precipitate. The solid was collected and dried in vacuum to give the title compound as citric acid salt. The title compound is C₂₅H₂₈ClN₇O₂, LC-MS (ESI, m/z): 492.4 [M−H]⁻.

### Example 2

(S)-(1-((2′-(2H-tetrazol-5-yl)biphenyl-4-yl)methyl)-2-butyl-4-chloro-1H-imidazol-5-yl)methyl pyrroldine-2-carboxylate. The title compound was prepared in accordance with General Procedure with one of the starting material being N-(tert-butoxycarbonyl)-L-proline. The compound is C₂₇H₃₀ClN₇O₂, LC-MS (ESI, m/z): 520.6 [M+H]⁺.

### Example 3

(S)-(1-((2′-(2H-tetrazol-5-yl)biphenyl-4-yl)methyl)-2-butyl-4-chloro-1H-imidazol-5-yl)methyl 2-amino-3-phenylpropanoate. The title compound was prepared in accordance with General Procedure with one of the starting material being N-(tert-butoxycarbonyl)-L-phenylalanine. The compound is C₃₁H₃₂ClN₇O₂, LC-MS (ESI, m/z): 570.5 [M+H]⁺.

### Example 4

(S)-(1-((2′-(2H-tetrazol-5-yl)biphenyl-4-yl)methyl)-2-butyl-4-chloro-1H-imidazol-5-yl)methyl 2-amino-3-hydroxypropanoate. The title compound was prepared in accordance with General Procedure with one of the starting material being N-(tert-butoxycarbonyl)-L-serine. The compound is C₃₅H₂₈ClN₇O₃, LC-MS (ESI, m/z): 510.4 [M+H]⁺.

### Example 5

(2S,3S)-(1-((2′-(2H-tetrazol-5-yl)biphenyl-4-yl)methyl)-2-butyl-4-chloro-1H-imidazol-5-yl)methyl 2-amino-3-hydroxybutanoate. The title compound was prepared in accordance with General Procedure with one of the starting material being N-(tert-butoxycarbonyl)-L-threonine. The compound is C₃₆H₃₀ClN₇O₃, LC-MS (ESI, m/z): 524.5 [M+H]⁺.

### Example 6

(S)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate. The title compound was prepared in accordance with General Procedure with one of the starting material being N-(tert-butoxycarbonyl)-L-tyrosine. The compound is C₃₁H₃₂ClN₇O₃, LC-MS (ESI, m/z): 586.4 [M+H]⁺.

### Example 7

(S)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate. The title compound was prepared in accordance with General Procedure with one of the starting material being N-(tert-butoxycarbonyl)-L-tryptophan. The compound is C₃₃H₃₃ClN₈O₂, LC-MS (ESI, m/z): 609.4 [M+H]⁺.

### Example 8

(S)-(1-((2′-(2H-tetrazol-5-yl)biphenyl-4-yl)methyl)-2-butyl-4-chloro-1H-imidazol-5-yl)methyl 2-amino-3-methylbutanoate. The title compound was prepared in accordance with General Procedure with one of the starting material being N-(tert-butoxycarbonyl)-L-valine. The compound is C₃₇H₃₂ClN₇O₂, LC-MS (ESI, m/z): 520.6 [M−H]⁻.

### Example 9

(2S,3R)-(1-((2′-(2H-tetrazol-5-yl)biphenyl-4-yl)methyl)-2-butyl-4-chloro-1H-imidazol-5-yl)methyl 2-amino-3-methylpentanoate. The title compound was prepared in accordance with General Procedure with one of the starting material being N-(tert-butoxycarbonyl)-L-isoleucine. The compound is C₂₈H₃₄ClN₇O₂, LC-MS (ESI, m/z): 534.6 [M−H]⁻.

### Example 10

(S)-(1-((2′-(2H-tetrazol-5-yl)biphenyl-4-yl)methyl)-2-butyl-4-chloro-1H-imidazol-5-yl)methyl 2,5-diamino-5-oxopentanoate. The title compound was prepared in accordance with General Procedure with one of the starting material being (S)-5-amino-2-(tert-butoxycarbonyl)amino-5-oxopentanoic acid. The compound is C₂₇H₃₁ClN₈O₃, LC-MS (ESI, m/z): 551.5 [M+H]⁺.

### Example 11

(S)-5-((1-((2′-(2H-tetrazol-5-yl)biphenyl-4-yl)methyl)-2-butyl-4-chloro-1H-imidazol-5-yl)methoxy)-4-amino-5-oxopentanoic acid. The title compound was prepared in accordance with General Procedure with one of the starting material being (S)-5-(tert-butoxy)-2-(tert-butoxycarbonyl)-amino 5-oxopentanoic acid. The compound is C₂₇H₃₀ClN₇O₄, LC-MS (ESI, m/z): 550.5 [M−H]⁻.

### Example 12

(S)-(1-((2′-(2H-tetrazol-5-yl)biphenyl-4-yl)methyl)-2-butyl-4-chloro-1H-imidazol-5-yl)methyl 2,6-diaminohexanoate. The title compound was prepared in accordance with General Procedure with one of the starting material being N,N′-di(tert-butoxycarbonyl)-L-lysine. The compound is C₂₈H₃₅ClN₈O₂, LC-MS (ESI, m/z): 551.5 [M+H]⁺.

### Example 13

(S)-(1-((2′-(2H-tetrazol-5-yl)biphenyl-4-yl)methyl)-2-butyl-4-chloro-1H-imidazol-5-yl)methyl 2-amino-4-(methylthio)butanoate. The title compound was prepared in accordance with General Procedure with one of the starting material being N-(tert-butoxycarbonyl)-L-methionine. The compound is C₂₇H₃₂ClN₇O₂S, LC-MS (ESI, m/z): 554.4 [M+H]⁺.

### Example 14

(S)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate. The title compound was prepared in accordance with General Procedure with one of the starting material being N-(tert-butoxycarbonyl)-L-histidine. The compound is C₂₈H₃₀ClN₉O₂, LC-MS (ESI, m/z): 560.3 [M+H]⁺.

### Example 15

(S)-(1-((2′-(2H-tetrazol-5-yl)biphenyl-4-yl)methyl)-2-butyl-4-chloro-1H-imidazol-5-yl)methyl 2-aminoacetate. The title compound was prepared in accordance with General Procedure with one of the starting material being N-(tert-butoxycarbonyl)-L-glycine. The compound is C₂₄H₂₆ClN₇O₂, LC-MS (ESI, m/z): 478.7 [M−H]⁻.

### Example 16

(S)-(1-((2′-(2H-tetrazol-5-yl)biphenyl-4-yl)methyl)-2-butyl-4-chloro-1H-imidazol-5-yl)methyl 2-amino-5-guanidinopentanoate. The title compound was prepared in accordance with General Procedure with one of the starting material being (S)-2-(tert-butoxycarbonyl)amino-5-guanidinopentanoic acid. The compound is C₂₈H₃₅ClN₁₀O₂, LC-MS (ESI, m/z): 579.3 [M+H]⁺.

### Example 17

(S)-(1-((2′-(2H-tetrazol-5-yl)biphenyl-4-yl)methyl)-2-butyl-4-chloro-1H-imidazol-5-yl)methyl 2,4-diamino-4-oxobutanoate. The title compound was prepared in accordance with General Procedure with one of the starting material being (S)-4-amino-2-(tert-butoxycarbonyl)amino-5-oxobutanoic acid. The compound is C₂₆H₂₉ClN₈O₃, LC-MS (ESI, m/z): 537.5 [M+H]⁺.

### Example 18

(S)-5-((1-((2′-(2H-tetrazol-5-yl)biphenyl-4-yl)methyl)-2-butyl-4-chloro-1H-imidazol-5-yl)methoxy)-3-amino-4-oxobutanoic acid. The title compound was prepared in accordance with General Procedure with one of the starting material being (S)-4-tert-butoxy-2-(tert-butoxycarbonyl)amino-4-oxobutanoic acid. The compound is C₂₆H₂₈ClN₇O₄, LC-MS (ESI, m/z): 536.4 [M−H]⁻.

Pharmacokinetic studies. The pharmacokinetics (PK) of compounds of formula (I) as well as Losartan were investigated in male Kumin mice. There were six mice per group and nine groups for this study. All mice were fasted overnight, following a single dose of 5.8 mg/kg referenced to the amount of metabolite, the compound of formula (2) from every compound of formula (1) studied. All mice were given dosing orally by gavage. The dosing vehicle used was propyleneglycol:water (1:1). The mice were fed 4 h post dose. Blood samples were collected at 0, 15, 30, 45 min, 1, 2, 4, 7, 10 and 15 h oral dosing. Approximately 0.3-0.5 ml of blood was collected from the jugular vein in tubes containing EDTA, and plasma was obtained by centrifugation. Plasma samples were stored at −20° C. until analysis. Samples were analyzed for the compound of formula (II), (1-((2′-(2H-tetrazol-5-yl)biphenyl-4-yl)methyl)-2-butyl-4-chloro-1H-imidazol-5-yl)methanol, by LC/MS/MS.

Compound (1) provided that Maximum Blood Concentration (Cₘₐₓ), Area Under the Curve (AUC), Bioavailability, and Half-time (T_{1/2}) of compound (II) in the PK study are 0.76 uM, 406 uM, 24%, and 2.78 h respectively.

Compound (92) provided that Maximum Blood Concentration (Cₘₐₓ) Area Under the Curve (AUC), Bioavailability, and Half-time (T_{1/2}) of compound (II) in the PK study are 0.644 uM, 342 uM, 20%, and 2.0 h respectively.

Compound (94) provided that Maximum Blood Concentration (Cₘₐₓ) Area Under the Curve (AUC), Bioavailability, and Half-time (T_{1/2}) of compound (II) in the PK study are 0.399 uM, 365 uM, 22%, and 33.2 h respectively.

Compound (104) provided that Maximum Blood Concentration (Cₘₐₓ), Area Under the Curve (AUC), Bioavailability, and Half-time (T_{1/2}) of compound (II) in the PK study are 0.751 uM, 482 uM, 29%, and 1.9 h respectively.

Compound (106) provided that Maximum Blood Concentration (Cₘₐₓ), Area Under the Curve (AUC), Bioavailability, and Half-time (T_{1/2}) of compound (II) in the PK study are 1.05 uM, 560 uM, 34%, and 12.5 h respectively.

Compound (109) provided that Maximum Blood Concentration (Cₘₐₓ), Area Under the Curve (AUC), Bioavailability, and Half-time (T_{1/2}) of compound (II) in the PK study are 0.214 uM, 209 uM, 13%, and 1.8 h respectively.

Compound (112) provided that Maximum Blood Concentration (Cₘₐₓ), Area Under the Curve (AUC), Bioavailability, and Half-time (T_{1/2}) of compound (II) in the PK study are 0.359 uM, 254 uM, 15%, and 8.2 h respectively.

Compound (114) provided that Maximum Blood Concentration (Cₘₐₓ), Area Under the Curve (AUC), Bioavailability, and Half-time (T_{1/2}) of compound (II) in the PK study are 0.333 uM, 209 uM, 13%, and 3.9 h respectively.

Compound (118) provided that Maximum Blood Concentration (Cₘₐₓ), Area Under the Curve (AUC), Bioavailability, and Half-time (T_{1/2}) of compound (II) in the PK study are 0.504 uM, 318 uM, 19%, and 1.9 h respectively.

Compound (134) provided that Maximum Blood Concentration (Cₘₐₓ), Area Under the Curve (AUC), Bioavailability, and Half-time (T_{1/2}) of compound (II) in the PK study are 0.544 uM, 532 uM, 33%, and 4.5 h respectively.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

1. A compound of Formula (I), or optical isomer, enantiomer, stereoisomer, solvate, hydrate, polymorph, or pharmaceutically acceptable salt thereof: 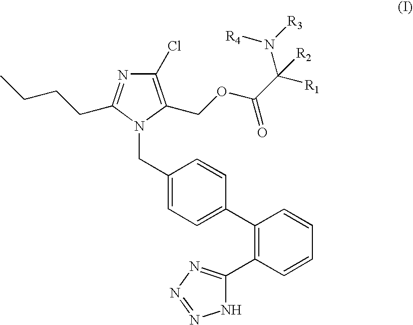
wherein R₁, R₂, R₃ and R₄ are each independently selected from, or one or more together comprise, H, and alkyl, alkenyl, aryl, heteroaryl, cycloalkyl, and heterocycloalkyl groups which are optionally substituted with one or more substituent selected from acyl, alkylamino, amino, aminocabonyl, acylamino, acyloxy, carboalkoxy, carboxy, carboxyamido, hydroxyl, thio, C₁₋₅ alkyl, C₂₋₄ alkenyl, C₃₋₆ cycloalkyl, C₃₋₆ heterocycloalkyl, C₆ aryl, C₅₋₈ heteroaryl, alkoxy, aryloxy, oxo, and guanidine.

2. The compound of claim 1, wherein R₁, R₂, R₃ and R₄ are each independently selected from, or one or more together comprise, H, C₁₋₈ alkyl, C₁₋₈ alkenyl, C₆₋₁₀ aryl, C₅₋₁₀ heteroaryl, C₃₋₁₀ cycloalkyl, and C₃₋₁₀ heterocycloalkyl.

3. The compound of claim 2, wherein R₁, R₂, R₃ and R₄ are each independently selected from, or one or more together comprise, H or C₁₋₆ alkyl.

4. The compound of claim 3, wherein C₁₋₆ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidino.

5. The compound of claim 2, wherein, R₁ and R₂ together comprise C₃₋₆ cycloalkyl or C₃₋₆ heterocycloalkyl when R₃ and R₄ are each independently selected from, or together comprise, H or C₁₋₆ alkyl.

6. The compound of claim 5, wherein C₁₋₆ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidine.

7. The compound of claim 5, wherein R₁ and R₂ together comprise cyclopropyl, cyclobutyl, cyclopentyl, or cyclohexyl, and optionally C₁₋₆ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidine.

8. The compound of claim 2, wherein R₃ and R₄ together comprise C₃₋₆ heterocycloalkyl when R₁ and R₂ are each independently selected from, or together comprise, H or C₁₋₆ alkyl.

9. The compound of claim 8, wherein C₁₋₆ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidine.

10. The compound of claim 8, wherein R₃ and R₄ together comprise aziridinyl, azetidinyl, pyrrolidinyl, or piperidinyl and optionally, C₁₋₆ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidine.

11. The compound of claim 1, wherein, when R₁, R₃ and R₄ are each H, R₂ is selected from H, CH₃, —CH₂CH₃, —CH(R₅)R₆, and —CH₂CH(R₅)R₆, wherein R₅ and R₆ are independently selected from C₁₋₃ alkyl.

12. The compound of claim 11, wherein C₁₋₃ alkyl is selected from methyl, ethyl, n-propyl, isopropyl, n-butyl, isobutyl, sec-butyl, tert-butyl, n-pentyl, isopentyl, neopentyl, n-hexyl, isohexyl, 1,1-dimethylbutyl, 2,2-dimethylbutyl, 3,3-dimethylbutyl, 2-ethylpropyl which are optionally substituted with one or more substituent selected from amino, aminocabonyl, acylamino, acyloxy, carbamoyl, carboalkoxy, carboxy, carboxyamido, hydroxyl, thio, alkylthio, alkoxy, aryloxy, oxo, and guanidino.

13. The compound of claim 1, wherein R₁, R₃ and R₄ are each H. and R₂ is selected from H, —CH₃, —CH₂CH₃, —CH(CH₃)CH₃, —CH₂CH(CH₃)CH₃, —CH(CH₃)CH₂CH₃, —CH₂CH₂CONH₂, —CH₂CONH₂, —CH₂CH₂COOH, —CH₂COOH, —SCH₃, —CH₂SH, —CH₂OH, —CH(OH)CH₃, —CH₂CH₂SCH₃, —CH₂C₆H₅, —CH₂C₆H₄(OH), —CH₂CH₂CH₂CH₂NH₂, 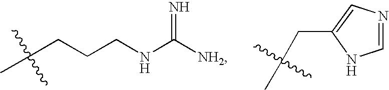
and

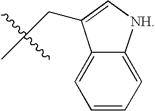

14. A pharmaceutical composition comprising a pharmaceutically acceptable carrier and a therapeutically effective amount of a compound of Formula (I) of claim 1.

15. A method for treating disorders selected from hypertension, congestive heart failure, and angina, comprising administering to a patient in need thereof a therapeutically effective amount of a compound of a compound of Formula (I), or optical isomer, enantiomer, stereoisomer, solvate, hydrate, polymorph, or pharmaceutically acceptable salt thereof: 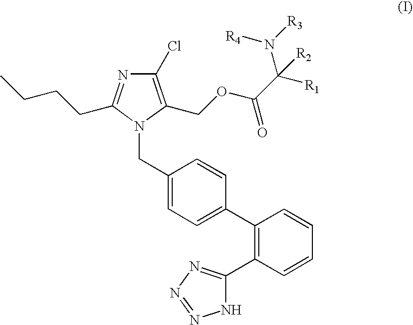
wherein R₁, R₂, R₃ and R₄ are each independently selected from, or one or more together comprise, H, and alkyl, alkenyl, aryl, heteroaryl, cycloalkyl, and heterocycloalkyl groups which are optionally substituted with one or more substituent selected from acyl, alkylamino, amino, aminocabonyl, acylamino, acyloxy, carboalkoxy, carboxy, carboxyamido, hydroxyl, thio, C₁₋₅ alkyl, C₂₋₄ alkenyl, C₃₋₆ cycloalkyl, C₃₋₆ heterocycloalkyl, C₆ aryl, C₅₋₈ heteroaryl, alkoxy, aryloxy, oxo, and guanidine.

16. A process of preparing a compound of Formula (I), or optical isomer, enantiomer, stereoisomer, solvate, hydrate, polymorph, or pharmaceutically acceptable salt thereof: 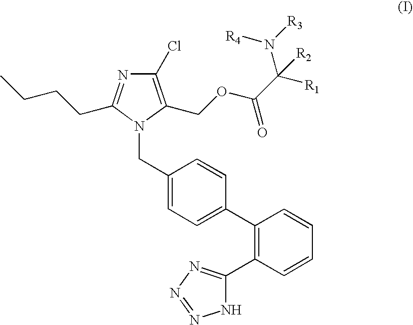
wherein R₁, R₂, R₃ and R₄ are each independently selected from, or one or more together comprise, H, and alkyl, alkenyl, aryl, heteroaryl, cycloalkyl, and heterocycloalkyl groups which are optionally substituted with one or more substituent selected from acyl, alkylamino, amino, aminocabonyl, acylamino, acyloxy, carboalkoxy, carboxy, carboxyamido, hydroxyl, thio, C₁₋₅ alkyl, C₂₋₄ alkenyl, C₃₋₆ cycloalkyl, C₃₋₆ heterocycloalkyl, C₆ aryl, C₅₋₈ heteroaryl, alkoxy, aryloxy, oxo, and guanidine,
comprising reacting by esterification a compound of Formula II with a compound of Formula III:

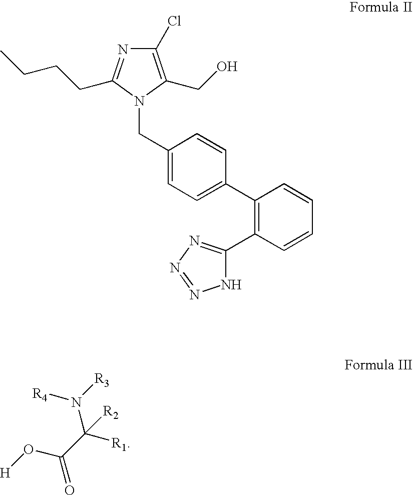

17. The compound of claim 1 selected from:
(S)-(1-((2′-(2H-tetrazol-5-yl)biphenyl-4-yl)methyl)-2-butyl-4-chloro-1H-imidazol-5-yl)methyl pyrroldine-2-carboxylate;
(R)-(1-((2′-(2H-tetrazol-5-yl)biphenyl-4-yl)methyl)-2-butyl-4-chloro-1H-imidazol-5-yl)methyl pyrroldine-2-carboxylate;
(S)-(1-((2′-(2H-tetrazol-5-yl)biphenyl-4-yl)methyl)-2-butyl-4-chloro-1H-imidazol-5-yl)methyl piperidine-2-carboxylate;
(R)-(1-((2′-(2H-tetrazol-5-yl)biphenyl-4-yl)methyl)-2-butyl-4-chloro-1H-imidazol-5-yl)methyl piperidine-2-carboxylate;
(S)-(1-((2′-(2H-tetrazol-5-yl)biphenyl-4-yl)methyl)-2-butyl-4-chloro-1H-imidazol-5-yl)methyl azepane-2-carboxylate;
(R)-(1-((2′-(2H-tetrazol-5-yl)biphenyl-4-yl)methyl)-2-butyl-4-chloro-1H-imidazol-5-yl)methyl azepane-2-carboxylate;
(1-((2′-(2H-tetrazol-5-yl)biphenyl-4-yl)methyl)-2-butyl-4-chloro-1H-imidazol-5-yl)methyl 1-aminocyclopentanecarboxylate;
(1-((2′-(2H-tetrazol-5-yl)biphenyl-4-yl)methyl)-2-butyl-4-chloro-1H-imidazol-5-yl)methyl (1-dimethylamino)cyclopentanecarboxylate;
(1-((2′-(2H-tetrazol-5-yl)biphenyl-4-yl)methyl)-2-butyl-4-chloro-1H-imidazol-5-yl)methyl (1-methylamino)cyclopentanecarboxylate;
(1-((2′-(2H-tetrazol-5-yl)biphenyl-4-yl)methyl)-2-butyl-4-chloro-1H-imidazol-5-yl)methyl (1-dimethylamino)cyclohexanecarboxylate;
(1-((2′-(2H-tetrazol-5-yl)biphenyl-4-yl)methyl)-2-butyl-4-chloro-1H-imidazol-5-yl)methyl (1-methylamino)cyclohexanecarboxylate;
1-((2′-(2H-tetrazol-5-yl)biphenyl-4-yl)methyl)-2-butyl-4-chloro-1H-imidazol-5-yl)methyl 2-methyl-2-(pyrrolidin-1-yl)propanoate;
(S)-(1-((2′-(2H-tetrazol-5-yl)biphenyl-4-yl)methyl)-2-butyl-4-chloro-1H-imidazol-5-yl)methyl 2-(pyrrolidin-1-yl)propanoate;
(R)-(1-((2′-(2H-tetrazol-5-yl)biphenyl-4-yl)methyl)-2-butyl-4-chloro-1H-imidazol-5-yl)methyl 2-(pyrrolidin-1-yl)propanoate;
(1-((2′-(2H-tetrazol-5-yl)biphenyl-4-yl)methyl)-2-butyl-4-chloro-1H-imidazol-5-yl)methyl 2-(dimethylamino)acetate;
(1-((2′-(2H-tetrazol-5-yl)biphenyl-4-yl)methyl)-2-butyl-4-chloro-1H-imidazol-5-yl)methyl 2-(methylamino)acetate;
(S)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
(R)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
(S)-(1-((2′-(2H-tetrazol-5-yl)biphenyl-4-yl)methyl)-2-butyl-4-chloro-1H-imidazol-5-yl)methyl 5-guanidino-2-(methylamino)pentanoate;
(S)-(1-((2′-(2H-tetrazol-5-yl)biphenyl-4-yl)methyl)-2-butyl-4-chloro-1H-imidazol-5-yl)methyl 2-(dimethylamino)-5-guanidinopentanoate;
(R)-(1-((2′-(2H-tetrazol-5-yl)biphenyl-4-yl)methyl)-2-butyl-4-chloro-1H-imidazol-5-yl)methyl 2-(dimethylamino)-5-guanidinopentanoate;
(S)-((1-((2′-(2H-tetrazol-5-yl)biphenyl-4-yl)methyl)-2-butyl-4-chloro-1H-imidazol-5-yl)methoxy)-3-(methylamino)-4-oxobutanoic acid;
(R)-((1-((2′-(2H-tetrazol-5-yl)biphenyl-4-yl)methyl)-2-butyl-4-chloro-1H-imidazol-5-yl)methoxy)-3-(methylamino)-4-oxobutanoic acid;
(S)-((1-((2′-(2H-tetrazol-5-yl)biphenyl-4-yl)methyl)-2-butyl-4-chloro-1H-imidazol-5-yl)methoxy)-3-(dimethylamino)-4-oxobutanoic acid;
(R)-((1-((2′-(2H-tetrazol-5-yl)biphenyl-4-yl)methyl)-2-butyl-4-chloro-1H-imidazol-5-yl)methoxy)-3-(dimethylamino)-4-oxobutanoic acid;
(S)-1-((1-((2′-(2H-tetrazol-5-yl)biphenyl-4-yl)methyl)-2-butyl-4-chloro-1H-imidazol-5-yl)methyl)-4-ethyl 2-(methylamino)succinate;
(R)-1-((1-((2′-(2H-tetrazol-5-yl)biphenyl-4-yl)methyl)-2-butyl-4-chloro-1H-imidazol-5-yl)methyl)-4-ethyl 2-(methylamino)succinate;
(S)-1-((1-((2′-(2H-tetrazol-5-yl)biphenyl-4-yl)methyl)-2-butyl-4-chloro-1H-imidazol-5-yl)methyl)-4-ethyl 2-(dimethylamino)succinate;
(R)-1-((1-((2′-(2H-tetrazol-5-yl)biphenyl-4-yl)methyl)-2-butyl-4-chloro-1H-imidazol-5-yl)methyl)-4-ethyl 2-(dimethylamino)succinate;
(S)-(1-((2′-(2H-tetrazol-5-yl)biphenyl-4-yl)methyl)-2-butyl-4-chloro-1H-imidazol-5-yl)methyl 3-mercapto-2-(methylamino)propanoate;
(R)-(1-((2′-(2H-tetrazol-5-yl)biphenyl-4-yl)methyl)-2-butyl-4-chloro-1H-imidazol-5-yl)methyl 3-mercapto-2-(methylamino)propanoate;
(S)-(1-((2′-(2H-tetrazol-5-yl)biphenyl-4-yl)methyl)-2-butyl-4-chloro-1H-imidazol-5-yl)methyl 3-mercapto-2-(dimethylamino)propanoate;
(R)-(1-((2′-(2H-tetrazol-5-yl)biphenyl-4-yl)methyl)-2-butyl-4-chloro-1H-imidazol-5-yl)methyl 3-mercapto-2-(dimethylamino)propanoate;
(S)-(1-((2′-(2H-tetrazol-5-yl)biphenyl-4-yl)methyl)-2-butyl-4-chloro-1H-imidazol-5-yl)methyl 4-amino-2-(methylamino)-4-oxobutanoate;
(R)-(1-((2′-(2H-tetrazol-5-yl)biphenyl-4-yl)methyl)-2-butyl-4-chloro-1H-imidazol-5-yl)methyl 4-amino-2-(methylamino)-4-oxobutanoate;
(S)-(1-((2′-(2H-tetrazol-5-yl)biphenyl-4-yl)methyl)-2-butyl-4-chloro-1H-imidazol-5-yl)methyl 4-amino-2-(dimethylamino)-4-oxobutanoate;
(R)-(1-((2′-(2H-tetrazol-5-yl)biphenyl-4-yl)methyl)-2-butyl-4-chloro-1H-imidazol-5-yl)methyl 4-amino-2-(dimethylamino)-4-oxobutanoate;
(S)-(1-((2′-(2H-tetrazol-5-yl)biphenyl-4-yl)methyl)-2-butyl-4-chloro-1H-imidazol-5-yl)methyl 5-amino-2-(methylamino)-5-oxopentanoate;
(R)-(1-((2′-(2H-tetrazol-5-yl)biphenyl-4-yl)methyl)-2-butyl-4-chloro-1H-imidazol-5-yl)methyl 5-amino-2-(methylamino)-5-oxopentanoate;
(S)-(1-((2′-(2H-tetrazol-5-yl)biphenyl-4-yl)methyl)-2-butyl-4-chloro-1H-imidazol-5-yl)methyl 5-amino-2-(dimethylamino)-5-oxopentanoate;
(R)-(1-((2′-(2H-tetrazol-5-yl)biphenyl-4-yl)methyl)-2-butyl-4-chloro-1H-imidazol-5-yl)methyl 5-amino-2-(dimethylamino)-5-oxopentanoate;
(S)-5-((1-((2′-(2H-tetrazol-5-yl)biphenyl-4-yl)methyl)-2-butyl-4-chloro-1H-imidazol-5-yl)methoxy)-4-(methylamino)-5-oxopentanoic acid;
(R)-5-((1-((2′-(2H-tetrazol-5-yl)biphenyl-4-yl)methyl)-2-butyl-4-chloro-1H-imidazol-5-yl)methoxy)-4-(methylamino)-5-oxopentanoic acid;
(S)-5-((1-((2′-(2H-tetrazol-5-yl)biphenyl-4-yl)methyl)-2-butyl-4-chloro-1H-imidazol-5-yl)methoxy)-4-(dimethylamino)-5-oxopentanoic acid;
(R)-5-((1-((2′-(2H-tetrazol-5-yl)biphenyl-4-yl)methyl)-2-butyl-4-chloro-1H-imidazol-5-yl)methoxy)-4-(dimethylamino)-5-oxopentanoic acid;
(S)-1-((1-((2′-(2H-tetrazol-5-yl)biphenyl-4-yl)methyl)-2-butyl-4-chloro-1H-imidazol-5-yl)methyl)-5-methyl 2-(methylamino)pentanedioate;
(R)-1-((1-((2′-(2H-tetrazol-5-yl)biphenyl-4-yl)methyl)-2-butyl-4-chloro-1H-imidazol-5-yl)methyl)-5-methyl 2-(methylamino)pentanedioate;
(S)-1-((1-((2′-(2H-tetrazol-5-yl)biphenyl-4-yl)methyl)-2-butyl-4-chloro-1H-imidazol-5-yl)methyl)-5-methyl 2-(dimethylamino)pentanedioate;
(R)-1-((1-((2′-(2H-tetrazol-5-yl)biphenyl-4-yl)methyl)-2-butyl-4-chloro-1H-imidazol-5-yl)methyl)-5-methyl 2-(dimethylamino)pentanedioate;
(S)-(1-((2′-(2H-tetrazol-5-yl)biphenyl-4-yl)methyl)-2-butyl-4-chloro-1H-imidazol-5-yl)methyl 3-(1H-imidazol-2-yl)-2-(methylamino)propanoate;
(R)-(1-((2′-(2H-tetrazol-5-yl)biphenyl-4-yl)methyl)-2-butyl-4-chloro-1H-imidazol-5-yl)methyl 3-(1H-imidazol-2-yl)-2-(methylamino)propanoate;
(S)-(1-((2′-(2H-tetrazol-5-yl)biphenyl-4-yl)methyl)-2-butyl-4-chloro-1H-imidazol-5-yl)methyl 2-(dimethylamino)-3-(1H-imidazol-2-yl)propanoate;
(R)-(1-((2′-(2H-tetrazol-5-yl)biphenyl-4-yl)methyl)-2-butyl-4-chloro-1H-imidazol-5-yl)methyl 2-(dimethylamino)-3-(1H-imidazol-2-yl)propanoate;
(2S,3S)-(1-((2′-(2H-tetrazol-5-yl)biphenyl-4-yl)methyl)-2-butyl-4-chloro-1H-imidazol-5-yl)methyl 3-methyl-2-(methylamino)pentanoate;
(2R,3S)-(1-((2′-(2H-tetrazol-5-yl)biphenyl-4-yl)methyl)-2-butyl-4-chloro-1H-imidazol-5-yl)methyl 3-methyl-2-(methylamino)pentanoate;
(2S,3S)-(1-((2′-(2H-tetrazol-5-yl)biphenyl-4-yl)methyl)-2-butyl-4-chloro-1H-imidazol-5-yl)methyl 2-(dimethylamino)-3-methyl-pentanoate;
(2R,3S)-(1-((2′-(2H-tetrazol-5-yl)biphenyl-4-yl)methyl)-2-butyl-4-chloro-1H-imidazol-5-yl)methyl 2-(dimethylamino)-3-methyl-pentanoate;
(S)-(1-((2′-(2H-tetrazol-5-yl)biphenyl-4-yl)methyl)-2-butyl-4-chloro-1H-imidazol-5-yl)methyl 6-amino-2-(methylamino)hexanoate;
(R)-(1-((2′-(2H-tetrazol-5-yl)biphenyl-4-yl)methyl)-2-butyl-4-chloro-1H-imidazol-5-yl)methyl 6-amino-2-(methylamino)hexanoate;
(S)-(1-((2′-(2H-tetrazol-5-yl)biphenyl-4-yl)methyl)-2-butyl-4-chloro-1H-imidazol-5-yl)methyl 6-amino-2-(dimethylamino)hexanoate;
(R)-(1-((2′-(2H-tetrazol-5-yl)biphenyl-4-yl)methyl)-2-butyl-4-chloro-1H-imidazol-5-yl)methyl 6-amino-2-(dimethylamino)hexanoate;
(S)-(1-((2′-(2H-tetrazol-5-yl)biphenyl-4-yl)methyl)-2-butyl-4-chloro-1H-imidazol-5-yl)methyl 2-(methylamino)-4-(methylthio)butanoate;
(R)-(1-((2′-(2H-tetrazol-5-yl)biphenyl-4-yl)methyl)-2-butyl-4-chloro-1H-imidazol-5-yl)methyl 2-(methylamino)-4-(methylthio)butanoate;
(S)-(1-((2′-(2H-tetrazol-5-yl)biphenyl-4-yl)methyl)-2-butyl-4-chloro-1H-imidazol-5-yl)methyl 2-(dimethylamino)-4-(methylthio)butanoate;
(R)-(1-((2′-(2H-tetrazol-5-yl)biphenyl-4-yl)methyl)-2-butyl-4-chloro-1H-imidazol-5-yl)methyl 2-(dimethylamino)-4-(methylthio)butanoate;
(S)-(1-((2′-(2H-tetrazol-5-yl)biphenyl-4-yl)methyl)-2-butyl-4-chloro-1H-imidazol-5-yl)methyl 2-(methylamino)-3-phenylpropanoate;
(R)-(1-((2′-(2H-tetrazol-5-yl)biphenyl-4-yl)methyl)-2-butyl-4-chloro-1H-imidazol-5-yl)methyl 2-(methylamino)-3-phenylpropanoate;
(S)-(1-((2′-(2H-tetrazol-5-yl)biphenyl-4-yl)methyl)-2-butyl-4-chloro-1H-imidazol-5-yl)methyl 2-(dimethylamino)-3-phenylpropanoate;
(R)-(1-((2′-(2H-tetrazol-5-yl)biphenyl-4-yl)methyl)-2-butyl-4-chloro-1H-imidazol-5-yl)methyl 2-(dimethylamino)-3-phenylpropanoate;
(S)-(1-((2′-(2H-tetrazol-5-yl)biphenyl-4-yl)methyl)-2-butyl-4-chloro-1H-imidazol-5-yl)methyl 1-methylpyrrolidine-2-carboxylate;
(R)-(1-((2′-(2H-tetrazol-5-yl)biphenyl-4-yl)methyl)-2-butyl-4-chloro-1H-imidazol-5-yl)methyl 1-methylpyrrolidine-2-carboxylate;
(S)-(1-((2′-(2H-tetrazol-5-yl)biphenyl-4-yl)methyl)-2-butyl-4-chloro-1H-imidazol-5-yl)methyl 3-hydroxy-2-(methylamino)propanoate;
(R)-(1-((2′-(2H-tetrazol-5-yl)biphenyl-4-yl)methyl)-2-butyl-4-chloro-1H-imidazol-5-yl)methyl 3-hydroxy-2-(methylamino)propanoate;
(S)-(1-((2′-(2H-tetrazol-5-yl)biphenyl-4-yl)methyl)-2-butyl-4-chloro-1H-imidazol-5-yl)methyl 2-(dimethylamino)-3-hydroxypropanoate;
(R)-(1-((2′-(2H-tetrazol-5-yl)biphenyl-4-yl)methyl)-2-butyl-4-chloro-1H-imidazol-5-yl)methyl 2-(dimethylamino)-3-hydroxypropanoate;
(S)-(1-((2′-(2H-tetrazol-5-yl)biphenyl-4-yl)methyl)-2-butyl-4-chloro-1H-imidazol-5-yl)methyl 3-hydroxy-2-(methylamino)butanoate;
(R)-(1-((2′-(2H-tetrazol-5-yl)biphenyl-4-yl)methyl)-2-butyl-4-chloro-1H-imidazol-5-yl)methyl 3-hydroxy-2-(methylamino)butanoate;
(S)-(1-((2′-(2H-tetrazol-5-yl)biphenyl-4-yl)methyl)-2-butyl-4-chloro-1H-imidazol-5-yl)methyl 2-(dimethylamino)-3-hydroxybutanoate;
(R)-(1-((2′-(2H-tetrazol-5-yl)biphenyl-4-yl)methyl)-2-butyl-4-chloro-1H-imidazol-5-yl)methyl 2-(dimethylamino)-3-hydroxybutanoate;
(S)-(1-((2′-(2H-tetrazol-5-yl)biphenyl-4-yl)methyl)-2-butyl-4-chloro-1H-imidazol-5-yl)methyl 3-(4-hydroxyphenyl)-2-(methylamino)propanoate;
(R)-(1-((2′-(2H-tetrazol-5-yl)biphenyl-4-yl)methyl)-2-butyl-4-chloro-1H-imidazol-5-yl)methyl 3-(4-hydroxyphenyl)-2-(methylamino)propanoate;
(S)-(1-((2′-(2H-tetrazol-5-yl)biphenyl-4-yl)methyl)-2-butyl-4-chloro-1H-imidazol-5-yl)methyl 2-(dimethylamino)-3-(4-hydroxyphenyl)propanoate;
(R)-(1-((2′-(2H-tetrazol-5-yl)biphenyl-4-yl)methyl)-2-butyl-4-chloro-1H-imidazol-5-yl)methyl 2-(dimethylamino)-3-(4-hydroxyphenyl)propanoate;
(S)-(1-((2′-(2H-tetrazol-5-yl)biphenyl-4-yl)methyl)-2-butyl-4-chloro-1H-imidazol-5-yl)methyl 3-methyl-2-(methylamino)butanoate;
(R)-(1-((2′-(2H-tetrazol-5-yl)biphenyl-4-yl)methyl)-2-butyl-4-chloro-1H-imidazol-5-yl)methyl 3-methyl-2-(methylamino)butanoate;
(S)-(1-((2′-(2H-tetrazol-5-yl)biphenyl-4-yl)methyl)-2-butyl-4-chloro-1H-imidazol-5-yl)methyl 2-(dimethylamino)-3-methylbutanoate;
(R)-(1-((2′-(2H-tetrazol-5-yl)biphenyl-4-yl)methyl)-2-butyl-4-chloro-1H-imidazol-5-yl)methyl 2-(dimethylamino)-3-methylbutanoate;
(S)-(1-((2′-(2H-tetrazol-5-yl)biphenyl-4-yl)methyl)-2-butyl-4-chloro-1H-imidazol-5-yl)methyl 3-(1H-indol-3-yl)-2-(methylamino)propanoate;
(R)-(1-((2′-(2H-tetrazol-5-yl)biphenyl-4-yl)methyl)-2-butyl-4-chloro-1H-imidazol-5-yl)methyl 3-(1H-indol-3-yl)-2-(methylamino)propanoate;
(S)-(1-((2′-(2H-tetrazol-5-yl)biphenyl-4-yl)methyl)-2-butyl-4-chloro-1H-imidazol-5-yl)methyl 2-(dimethylamino)-3-(1H-indol-3-yl)propanoate;
(R)-(1-((2′-(2H-tetrazol-5-yl)biphenyl-4-yl)methyl)-2-butyl-4-chloro-1H-imidazol-5-yl)methyl 2-(dimethylamino)-3-(1H-indol-3-yl)propanoate;
(S)-(1-((2′-(2H-tetrazol-5-yl)biphenyl-4-yl)methyl)-2-butyl-4-chloro-1H-imidazol-5-yl)methyl 2-amino-3-phenylpropanoate;
(R)-(1-((2′-(2H-tetrazol-5-yl)biphenyl-4-yl)methyl)-2-butyl-4-chloro-1H-imidazol-5-yl)methyl 2-amino-3-phenylpropanoate;
(S)-(1-((2′-(2H-tetrazol-5-yl)biphenyl-4-yl)methyl)-2-butyl-4-chloro-1H-imidazol-5-yl)methylpyrrolidine-2-carboxylate;
(R)-(1-((2′-(2H-tetrazol-5-yl)biphenyl-4-yl)methyl)-2-butyl-4-chloro-1H-imidazol-5-yl)methylpyrrolidine-2-carboxylate;
(S)-(1-((2′-(2H-tetrazol-5-yl)biphenyl-4-yl)methyl)-2-butyl-4-chloro-1H-imidazol-5-yl)methyl 2-amino-3-hydroxypropanoate;
(R)-(1-((2′-(2H-tetrazol-5-yl)biphenyl-4-yl)methyl)-2-butyl-4-chloro-1H-imidazol-5-yl)methyl 2-amino-3-hydroxypropanoate;
(2S,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
(2R,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
(S)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
(R)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
(S)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
(R)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
(S)-(1-((2′-(2H-tetrazol-5-yl)biphenyl-4-yl)methyl)-2-butyl-4-chloro-1H-imidazol-5-yl)methyl 2-amino-3-methylbutanoate;
(R)-(1-((2′-(2H-tetrazol-5-yl)biphenyl-4-yl)methyl)-2-butyl-4-chloro-1H-imidazol-5-yl)methyl 2-amino-3-methylbutnoate;
(2S,3R)-(1-((2′-(2H-tetrazol-5-yl)biphenyl-4-yl)methyl)-2-butyl-4-chloro-1H-imidazol-5-yl)methyl 2-amino-3-methylpentanoate;
(2R,3R)-(1-((2′-(2H-tetrazol-5-yl)biphenyl-4-yl)methyl)-2-butyl-4-chloro-1H-imidazol-5-yl)methyl 2-amino-3-methylpentanoate;
(S)-(1-((2′-(2H-tetrazol-5-yl)biphenyl-4-yl)methyl)-2-butyl-4-chloro-1H-imidazol-5-yl)methyl 2,5-diamino-5-oxopentanoate;
(R)-(1-((2′-(2H-tetrazol-5-yl)biphenyl-4-yl)methyl)-2-butyl-4-chloro-1H-imidazol-5-yl)methyl 2,5-diamino-5-oxopentanoate;
(S)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
(R)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
(S)-(1-((2′-(2H-tetrazol-5-yl)biphenyl-4-yl)methyl)-2-butyl-4-chloro-1H-imidazol-5-yl)methyl 2,6-diaminohexanoate;
(R)-(1-((2′-(2H-tetrazol-5-yl)biphenyl-4-yl)methyl)-2-butyl-4-chloro-1H-imidazol-5-yl)methyl 2,6-diaminohexanoate;
(S)-(1-((2′-(2H-tetrazol-5-yl)biphenyl-4-yl)methyl)-2-butyl-4-chloro-1H-imidazol-5-yl)methyl 2-amino-4-(methylthio)butanoate;
(R)-(1-((2′-(2H-tetrazol-5-yl)biphenyl-4-yl)methyl)-2-butyl-4-chloro-1H-imidazol-5-yl)methyl 2-amino-4-(methylthio)butanoate;
(S)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
(R)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
(S)-(1-((2′-(2H-tetrazol-5-yl)biphenyl-4-yl)methyl)-2-butyl-4-chloro-1H-imidazol-5-yl)methyl 2-aminoacetate;
(R)-(1-((2′-(2H-tetrazol-5-yl)biphenyl-4-yl)methyl)-2-butyl-4-chloro-1H-imidazol-5-yl)methyl 2-aminoacetate;
(S)-(1-((2′-(2H-tetrazol-5-yl)biphenyl-4-yl)methyl)-2-butyl-4-chloro-1H-imidazol-5-yl)methyl 2-aminopropanoate;
(R)-(1-((2′-(2H-tetrazol-5-yl)biphenyl-4-yl)methyl)-2-butyl-4-chloro-1H-imidazol-5-yl)methyl 2-aminopropanoate;
(S)-(1-((2′-(2H-tetrazol-5-yl)biphenyl-4-yl)methyl)-2-butyl-4-chloro-1H-imidazol-5-yl)methyl 2-amino-5-guanidinopentanoate;
(R)-(1-((2′-(2H-tetrazol-5-yl)biphenyl-4-yl)methyl)-2-butyl-4-chloro-1H-imidazol-5-yl)methyl 2-amino-5-guanidinopentanoate;
(S)-4-((1-((2′-(2H-tetrazol-5-yl)biphenyl-4-yl)methyl)-2-butyl-4-chloro-1H-imidazol-5-yl)methoxy)-3-amino-4-oxobutanoic acid;
(R)-4-((1-((2′-(2H-tetrazol-5-yl)biphenyl-4-yl)methyl)-2-butyl-4-chloro-1H-imidazol-5-yl)methoxy)-3-amino-4-oxobutanoic acid;
(S)-(1-((2′-(2H-tetrazol-5-yl)biphenyl-4-yl)methyl)-2-butyl-4-chloro-1H-imidazol-5-yl)methyl 2,4-diamino-4-oxobutanoate;
(R)-(1-((2′-(2H-tetrazol-5-yl)biphenyl-4-yl)methyl)-2-butyl-4-chloro-1H-imidazol-5-yl)methyl 2,4-diamino-4-oxobutanoate;
(S)-(1-((2′-(2H-tetrazol-5-yl)biphenyl-4-yl)methyl)-2-butyl-4-chloro-1H-imidazol-5-yl)methyl 2-amino-3-mercaptopropanoate;
(R)-(1-((2′-(2H-tetrazol-5-yl)biphenyl-4-yl)methyl)-2-butyl-4-chloro-1H-imidazol-5-yl)methyl 2-amino-3-mercaptopropanoate;
1-((2′-(2H-tetrazol-5-yl)biphenyl-4-yl)methyl)-2-butyl-4-chloro-1H-imidazol-5-yl)methyl 2-methyl-2-(methylamino)propanoate;
1-((2′-(2H-tetrazol-5-yl)biphenyl-4-yl)methyl)-2-butyl-4-chloro-1H-imidazol-5-yl)methyl 2-(dimethylamino)-2-methylpropanoate;
1-((2′-(2H-tetrazol-5-yl)biphenyl-4-yl)methyl)-2-butyl-4-chloro-1H-imidazol-5-yl)methyl 1-(methylamino)cyclopropanecarboxylate;
1-((2′-(2H-tetrazol-5-yl)biphenyl-4-yl)methyl)-2-butyl-4-chloro-1H-imidazol-5-yl)methyl 1-(dimethylamino)cyclopropanecarboxylate;
1-((2′-(2H-tetrazol-5-yl)biphenyl-4-yl)methyl)-2-butyl-4-chloro-1H-imidazol-5-yl)methyl 1-(methylamino)cyclobutanecarboxylate; and
1-((2′-(2H-tetrazol-5-yl)biphenyl-4-yl)methyl)-2-butyl-4-chloro-1H-imidazol-5-yl)methyl 1-(dimethylamino)cyclobutanecarboxylate.

18. The compound of claim 1 selected from:
(S)-(1-((2′-(2H-tetrazol-5-yl)biphenyl-4-yl)methyl)-2-butyl-4-chloro-1H-imidazol-5-yl)methyl 2-aminopropanoate;
(S)-(1-((2′-(2H-tetrazol-5-yl)biphenyl-4-yl)methyl)-2-butyl-4-chloro-1H-imidazol-5-yl)methyl pyrroldine-2-carboxylate;
(S)-(1-((2′-(2H-tetrazol-5-yl)biphenyl-4-yl)methyl)-2-butyl-4-chloro-1H-imidazol-5-yl)methyl 2-amino-3-phenylpropanoate;
(S)-(1-((2′-(2H-tetrazol-5-yl)biphenyl-4-yl)methyl)-2-butyl-4-chloro-1H-imidazol-5-yl)methyl 2-amino-3-hydroxypropanoate;
(2S,3S)-(1-((2′-(2H-tetrazol-5-yl)biphenyl-4-yl)methyl)-2-butyl-4-chloro-1H-imidazol-5-yl)methyl 2-amino-3-hydroxybutanoate;
(S)-(1-((2′-(2H-tetrazol-5-yl)biphenyl-4-yl)methyl)-2-butyl-4-chloro-1H-imidazol-5-yl)methyl 2-amino-3-(4-hydroxyphenyl)propanoate;
(S)-(1-((2′-(2H-tetrazol-5-yl)biphenyl-4-yl)methyl)-2-butyl-4-chloro-1H-imidazol-5-yl)methyl 2-amino-3-(1H-indol-3-yl)propanoate;
(S)-(1-((2′-(2H-tetrazol-5-yl)biphenyl-4-yl)methyl)-2-butyl-4-chloro-1H-imidazol-5-yl)methyl 2-amino-3-methylbutanoate;
(2S,3R)-(1-((2′-(2H-tetrazol-5-yl)biphenyl-4-yl)methyl)-2-butyl-4-chloro-1H-imidazol-5-yl)methyl 2-amino-3-methylpentanoate;
(S)-(1-((2′-(2H-tetrazol-5-yl)biphenyl-4-yl)methyl)-2-butyl-4-chloro-1H-imidazol-5-yl)methyl 2,5-diamino-5-oxopentanoate;
(S)-5-((1-((2′-(2H-tetrazol-5-yl)biphenyl-4-yl)methyl)-2-butyl-4-chloro-1H-imidazol-5-yl)methoxy)-4-amino-5-oxopentanoic acid;
(S)-(1-((2′-(2H-tetrazol-5-yl)biphenyl-4-yl)methyl)-2-butyl-4-chloro-1H-imidazol-5-yl)methyl 2,6-diaminohexanoate;
(S)-(1-((2′-(2H-tetrazol-5-yl)biphenyl-4-yl)methyl)-2-butyl-4-chloro-1H-imidazol-5-yl)methyl 2-amino-4-(methylthio)butanoate;
(S)-(1-((2′-(2H-tetrazol-5-yl)biphenyl-4-yl)methyl)-2-butyl-4-chloro-1H-imidazol-5-yl)methyl 2-amino-3-(1H-imidazol-2-yl)propanoate;
(S)-(1-((2′-(2H-tetrazol-5-yl)biphenyl-4-yl)methyl)-2-butyl-4-chloro-1H-imidazol-5-yl)methyl 2-aminoacetate;
(S)-(1-((2′-(2H-tetrazol-5-yl)biphenyl-4-yl)methyl)-2-butyl-4-chloro-1H-imidazol-5-yl)methyl 2-amino-5-guanidinopentanoate;
(S)-(1-((2′-(2H-tetrazol-5-yl)biphenyl-4-yl)methyl)-2-butyl-4-chloro-1H-imidazol-5-yl)methyl 2,4-diamino-4-oxobutanoate; and
(S)-5-((1-((2′-(2H-tetrazol-5-yl)biphenyl-4-yl)methyl)-2-butyl-4-chloro-1H-imidazol-5-yl)methoxy)-3-amino-4-oxobutanoic acid.

19. A pharmaceutical composition comprising a pharmaceutically acceptable carrier and a therapeutically effective amount of a compound of claim 17.

20. A pharmaceutical composition comprising a pharmaceutically acceptable carrier and a therapeutically effective amount of a compound of claim 18.
